(19) United States
(12) Patent Application Publication
(10) Pub. No.: US 2014/0199738 A1
(43) Pub. Date: Jul. 17, 2014

(54) METHOD FOR PREPARING VOLATILE FATTY ACIDS FROM THE PRE-TREATED EXTRACTS OF MARINE BIOMASS RESIDUE

(71) Applicants: Hee-Chul Woo, Busan (KR); Ho-Nam Chang, Seoul (KR); Yeong-Joong Jeon, Seoul (KR); Dong-Jin Suh, Seoul (KR); Byung-Soo Chun, Busan (KR); Kyeong-Keun Oh, Seongnam-si, Gyeonggi-do (KR); Kyoung-Heon Kim, Seoul (KR); Du-Woon Kim, Gwangju (KR); Jae-Hyung Choi, Busan (KR)

(72) Inventors: Hee-Chul Woo, Busan (KR); Ho-Nam Chang, Seoul (KR); Yeong-Joong Jeon, Seoul (KR); Dong-Jin Suh, Seoul (KR); Byung-Soo Chun, Busan (KR); Kyeong-Keun Oh, Seongnam-si, Gyeonggi-do (KR); Kyoung-Heon Kim, Seoul (KR); Du-Woon Kim, Gwangju (KR); Jae-Hyung Choi, Busan (KR)

(73) Assignee: Pukyong National University Industry- University Cooperation Foundation, Busan (KR)

(21) Appl. No.: 13/807,587

(22) PCT Filed: Dec. 16, 2011
(86) PCT No.: PCT/KR11/09701

§ 371 Date: Dec. 28, 2012

(30) Foreign Application Priority Data
Aug. 2, 2011 (KR) 10-2011-0077148

(51) Int. Cl. C12P 7/64

(52) U.S. Cl. CPC C12P 7/6409
USPC 435/134

(57) ABSTRACT

A method of preparing a volatile fatty acids (VFAs) is provided. More particularly, the method includes chemically or biologically pretreating a residue of algae to obtain an extract of the algae residue, filtering the extract of the algae residue and anaerobically fermenting the filtrate. The method of preparing VFAs can be used regardless of the kinds of algae, a process of producing VFAs can be simplified since there is no need to develop an enzyme for anaerobic fermentation, and a yield of VFAs can be increased since the VFAs can be produced from different kinds of organic components such as proteins or fats in addition to carbohydrates. Accordingly, the method can be useful in producing VFAs in an economical and efficient manner.

### REFERENCE TO RELATED APPLICATIONS

This application is a National Phase of International Application No. PCT/KR2011/009701, which was filed on Dec. 16, 2011, and which claims priority to and the benefit of Korean Patent Application No. 10-2011-0077148, filed on Aug. 2, 2011, and the disclosures of which are hereby incorporated herein by reference in their entireties.

### BACKGROUND

1. Technical Field

Example embodiments of the present invention relate in general to a method of preparing a volatile fatty acid, and more particularly, to a method of preparing a volatile fatty acid using a residue of algae (marine algae biomass) that remains after extracting or fractionating the algae with an organic solvent.

2. Related Art

Biofuel refers to a fuel obtained from a biomass. Such biofuel can be produced by subjecting a biomass to a thermochemical or biological method. A variety of biofuels can be produced using such a method. Among these, an anaerobic fermentation platform (or a volatile fatty acid (VFA) platform) is a method that includes mixing biomass with an anaerobic microorganism and incubating the anaerobic microorganism to degrade the biomass into a VFA mixture and optionally converting the VFA mixture into a gaseous fuel such as hydrogen to recover the gaseous fuel or converting the VFA mixture into a liquid fuel including an alcohol mixture using a hydrogenation reaction. The VFAs produced by such a VFA platform is composed of acetic acid (C2), propionic acid (C3), butyric acid (C4), etc., which are then converted into ethanol, propanol, butanol, etc., respectively, through the hydrogenation reaction. The MixAlco technology developed at Texas A&M University (US) is a representative method of producing a mixed alcohol from wood using the described-above VFA platform, and is now in final case study.

Biomasses are largely divided into three categories: first generation (sugar or starch biomasses), second generation (lignocellulosic biomass) and third generation (marine algae biomass). Each biomass can undergo a pretreatment process and a saccharification process, followed by a fermentation process to produce a biofuel. However, since the biomasses other than the lignocellulosic biomasses, that is, the sugar or starch biomasses, are used as food resources by human beings, there is a problem in that foods that should be used as an energy source are ineffective because the biofuel is produced at a relatively smaller amount than that of the biomass being consumed. On the other hand, the lignocellulosic biomass have a problem in that economical efficiency is lowered due to the additional processing costs required for a pretreatment process to remove lignin that is a component of lignocellulosic biomass. Also, such terrestrial biomasses have a problem in that environmental issues may be caused due to the use of fertilizers. In addition, in countries with limited land space such as the Republic of Korea, the soil is insufficient for producing the land biomasses.

Meanwhile, unlike the terrestrial biomasses, marine algae biomasses have advantages such as a better growth rate than the terrestrial biomass, no limit to a cultivation area due to the use of the vast ocean, and reduced use of expensive resources such as soil and fertilizers. A VFA platform using such an algae biomass has advantages in that it can convert organic components (proteins, lipids, etc.) other than carbohydrates into VFAs, does not require a sterilization process to incubate a strain, and there is no need to develop a certain enzyme. Also, it is known that, since the most of algae biomass does not include a lignin component, it takes a short period of time (e.g., from five to ten days) to produce VFAs. In addition, an enclosed reactor can be used to recover hydrogen produced during fermentation of a VFA, the recovered hydrogen can be easily used for a subsequent hydrogenation reaction, and it is easy to handle problems regarding a bad smell which is expected to be a problem of the VFA platform.

In spite of the above-described advantages, research on pretreatment and fermentation has hardly been conducted to produce a mixture of VFAs from algae by means of the VFA platform, and it is still difficult to secure the economical processing efficiency due to low yield in the VFA platform.

### SUMMARY

Accordingly, example embodiments of the present invention are provided to substantially obviate one or more problems due to limitations and disadvantages of the related art.

Example embodiments of the present invention provide a method of producing volatile fatty acids (VFAs) with high efficiency using a filtrate of an extract obtained by pretreating and extracting a residue of an algae biomass extract or fraction.

In some example embodiments, a method of preparing VFAs includes collecting a residue of algae that remains after extracting or fractionating the algae with an organic solvent, chemically or biologically pretreating the residue of algae to obtain an extract of the algae residue, filtering the extract of the algae residue to obtain a filtrate and anaerobically fermenting the filtrate.

### BRIEF DESCRIPTION OF DRAWINGS

Example embodiments of the present invention will become more apparent by describing in detail example embodiments of the present invention with reference to the accompanying drawings, in which:

FIG. 1 is a flowchart schematically showing a method of preparing volatile fatty acids (VFAs) according to Example embodiments of the present invention.

FIG. 2 is a diagram showing a fermentation apparatus used for anaerobic fermentation according to Example embodiments of the present invention.

FIG. 3 is a graph showing the compositions of VFAs according to kinds of algae.

FIG. 4 is a graph showing the compositions of VFAs according to pretreatment conditions 1 to 4 described in Example <2-3> of the present invention.

FIG. 5 is a graph showing the compositions of VFAs according to pretreatment conditions 5 to 8 described in Example <2-3> of the present invention.

FIG. 6 is a graph showing the compositions of VFAs according to pretreatment conditions 9 to 12 described in Example <2-3> of the present invention.

FIG. 7 is a graph showing the compositions of VFAs according to a change in anaerobic fermentation temperature.

FIG. 8 is a graph showing the compositions of VFAs according to a change in anaerobic fermentation acidity.

FIG. 9 is a graph showing the compositions of VFAs according to the kinds of methane production inhibitors added during anaerobic fermentation.

FIG. 10 is a graph showing the compositions of VFAs according to the kinds of medium compositions (medium 1 to medium 3) in which an anaerobic microorganism is incubated during anaerobic fermentation.

FIG. 11 is a graph showing the compositions of VFAs according to the kinds of medium compositions (media 4 and 5) in which an anaerobic microorganism is incubated during anaerobic fermentation.

FIG. 12 is a diagram showing a 300 L continuous anaerobic fermentation apparatus.

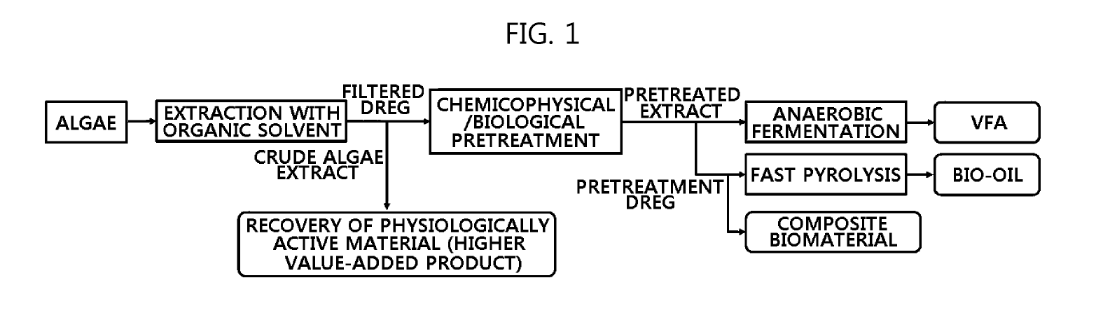

### DESCRIPTION OF EXAMPLE EMBODIMENTS

Example embodiments of the present invention are disclosed herein. However, specific structural and functional details disclosed herein are merely representative for purposes of describing example embodiments of the present invention, however, example embodiments of the present invention may be embodied in many alternate forms and should not be construed as limited to example embodiments of the present invention set forth herein.

Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present invention. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between”, “adjacent” versus “directly adjacent”, etc.).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should also be noted that in some alternative implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved.

Example embodiments of the present invention provide a method of preparing a volatile fatty acid (VFA) including: 1) collecting a residue of algae biomass that remains after extracting or fractionating the algae with an organic solvent, 2) chemically or biologically pretreating the residue of algae to obtain an extract of the algae residue, 3) filtering the extract of the algae residue to obtain a filtrate, and 4) anaerobically fermenting the filtrate.

Step 1) is to collect a dreg, for example, a residue of an algae extract or fraction, which remains after extracting or fractionating algae with an organic solvent, followed by extracting or fractionating soluble materials included in the algae. The method of preparing a VFA according to Example embodiments of the present invention does not use an extract or fraction obtained by primarily extracting or fractionating algae, but re-uses a waste material such as a dreg (residue) that remains after extracting or fractionating the algae so as to produce VFAs.

The algae in Step 1) may be macro-algae or micro-algae. The macro-algae may include brown algae, red algae or green algae, and the micro-algae may include Chlorella, Spirulina or Dunaliella sp. The brown algae may include, but is not limited to, Laminaria japonica, Undaria pinnatifida, Hizikia fusiforme, Sargassum fulvellum, Ecklonia stolonifera, Analipus japonicus, Chordaria flagelliformis, Ishige okamurae, Scytosiphon lomentaria, Endarachne binghamiae, Ecklonia cava, Costaria costata, Sargassum horneri, Sargassum thunbergii or Eisenia bicyclis. Also, the red algae may include, but is not limited to, Pachymeniopsis elliptica, Porphyra umbilicalis, Gelidium amansii, Cottonni, Pachymeniopsis lanceolata, Pachymeniopsis suborbiculata, Pterocladia tenuis, Acanthopeltis japonica, Gloiopeltis tenax, Chondrus ocellatus, Grateloupia elliptica, Hypnea sp., Ceramium sp., Ceramium boydenii, Chondracanthus tenellus, Graptoloidea sp., Pachymeniopsis elliptica or Gracilaria verrucosa, and the green algae may include, but is not limited to, Sphagnum sp., Spirogyra sp., Ulva pertusa, Codium fragile, Codium minus, Caulerpa sp., Nostoc sp., Ulva lactuca or Monostroma nitidum. In particular, the algae are preferably the brown algae, and more preferably L. japonica. According to Example embodiments of the present invention, it was confirmed that the brown algae L. japonica is most effectively used to produce VFAs.

The algae of Step 1) may be physically pretreated to enhance pretreatment efficiency by enlarging a reaction surface area during extraction or fractionation of algae. The physical pretreatment means that algae are ground or cut, and may be performed using all kinds of tools capable of grinding or cutting the algae. Especially, the physical pretreatment may be performed using a ball mill or a knife.

The residue of algae obtained in Step 1) is a dreg remaining after extraction or fractionation with the organic solvent, that is, a dreg of algae remaining after directly dissolving or separating soluble materials (i.e., materials soluble in an organic solvent) from algae directly or through respective operations and extracting or fractionating the soluble materials included in the algae. The organic solvent that may be used herein may include at least one selected from the group consisting of ethanol, n-hexane, dichloromethane, ethylacetate and n-butanol.

Steps 2) to 4) are to produce VFAs using the dreg remaining after extracting or fractionating the soluble materials in the organic solvent in Step 1). Unless explicitly described otherwise hereinafter, the term “residue of algae” or “algae residue” used herein refers to a “dreg remaining after primarily extracting or fractionating the algae obtained in Step 1) using an organic solvent.”

Step 2) is to pretreat the residue of algae so as to produce VFAs from the residue of algae obtained in Step 1). Here, the residue of algae may be chemically or biologically pretreated. In the pretreatment of the algae residue described above, materials used to produce the VFAs from the algae residue are extracted, thereby obtaining an extract of the algae residue.

The chemical pretreatment of Step 2) may be performed by treating the algae residue with at least one selected from the group consisting of an acid catalyst, an alkali catalyst and a supercritical fluid. The acid catalyst may be at least one selected from the group consisting of sulfuric acid, nitric acid, phosphoric acid, hydrochloric acid, perchloric acid, p-toluenesulfonic acid, methanesulfonic acid, formic acid, acetic acid, hydrofluoric acid, boric acid and a commercially available solid acid, the alkali catalyst may be at least one selected from the group consisting of potassium hydroxide, sodium hydroxide, calcium hydroxide, barium hydroxide, ammonium hydroxide and basic zeolite, and the supercritical fluid may be supercritical carbon dioxide or supercritical water. The pretreatment with the acid catalyst may be performed using a first acid catalyst treatment method in which a reaction is carried out in a batch-type reactor and a second acid catalyst treatment method in which a reaction is carried out in a reflux-type reactor. According to Example embodiments of the present invention, it is confirmed that the pretreatment using the first acid catalyst treatment method in which a reaction is carried out in a batch-type reactor is more effective in producing VFAs.

The biological pretreatment of Step 2) may be performed by treating the algae residue with a culture broth including a microorganism. The microorganism may be derived from abalone viscera or foreshore. The abalone viscera- or foreshore-derived microorganism shows halophilicity and has a characteristic of easily taking in and digesting the algae.

The filtration of Step 3) is to obtain an extract extracted from the algae residue, that is, only a liquid including materials that are derived from the algae residue and used to produce the VFAs. The filtrate obtained by the filtration may be used to produce the VFAs. That is, a solid product of the pretreated algae residue is not used, but a filtrate obtained by filtering the extract derived from the algae residue is used herein. The extract of the algae residue is a mixture including a carbohydrate such as a fermentable sugar (glucose, fucose, mannitol, etc.), a non-degradable sugar (alginate, uronic acid, etc.), a protein, a lipid and a compound whose molecular weight is reduced by the pretreatment as main components. The components constituting the extract of the algae residue described above may be used as a carbon source to produce the VFAs. Also, the solid product of the pretreated algae may be separately separated without use for production of VFAs, and may be used as a raw material such as a composite biomaterial, a natural fertilizer or bio-oil, which is used for a fast pyrolysis process. According to Example embodiments of the present invention, only volatile organic acids are not produced using the total solid algae as a raw source like technologies known in the art, but VFAs are produced from a residue of algae remaining after primary recovery (extraction or separation) of physiologically active materials from the algae and the pretreated solid product is used as a raw material such as a composite biomaterial, a natural fertilizer or bio-oil so that the use of an algae source can be maximized and the economical efficiency can be secured.

The filtrate of Step 4) preferably has a concentration of 6 g/L to 84 g/L, and more preferably a concentration of 18 g/L to 54 g/L. When the concentration of the filtrate is less than 6 g/L, it takes more expense to recover the produced VFAs due to their low concentration. On the other hand, when the concentration of the filtrate exceeds 84 g/L, a concentration of the produced VFAs is not increased any more, and thus the yield of the VFAs may be decreased with an increase in amount of an unreacted extract. When a concentration of the filtrate from the extract of the algae residue is out of a range of 6 g/L to 84 g/L, the concentration of the filtrate from the extract of the algae residue may be adjusted to this concentration range by diluting or concentrating the filtrate from the extract of the algae residue. According to Example embodiments of the present invention, it is confirmed that the VFAs may be most effectively produced when the extract filtrate is present within this concentration range.

The anaerobic fermentation of Step 4) may be performed using an anaerobic microorganism. When the filtrate obtained in Step 3) is fermented with an anaerobic microorganism, the filtrate is fermented under the anaerobic condition. The anaerobic microorganism may be an anaerobic fermentation strain widely used in the art. Here, the use of an anaerobic fermentation strain having salt tolerance is preferred. In general, the anaerobic fermentation strain may be isolated from the viscera or excretion of an herbivorous animal such as a cow or a goat, an acid fermentation tank for methane production, a methane fermentation tank, an anaerobic fermentation tank for food waste, and a site (such as foreshore) having a high activity of degrading organic matters. In particular, the anaerobic fermentation strain may be at least one selected from the group consisting of Clostridium sp., Acetogenium sp., Peptococcus sp., Acetobacterium sp., Pseudomonas sp. and Propionobacterium sp. The anaerobic microorganism may be introduced at a volume ratio of 1/30 to 1/10, based on the volume of the filtrate to be fermented. When an amount of the introduced anaerobic microorganism is less than a volume ratio of 1/30, it takes more expense to recover the produced VFAs due to their low concentration. On the other hand, when the amount of the introduced anaerobic microorganism exceeds a volume ratio of 1/10, a concentration of the produced VFAs is not increased any more, and thus the yield of the VFAs may be decreased with an increase in amount of an unreacted extract. In particular, according to Example embodiments of the present invention, it is confirmed that production of the VFAs is most effective when the anaerobic microorganism is introduced at a volume ratio of 1/15.

The anaerobic fermentation of Step 4) may be performed in the presence of a methane production inhibitor. The methane production inhibitor functions to prevent a decrease in concentration of the VFAs by preventing the VFAs from being converted into methane by a methane-producing microorganism in an anaerobic fermentation process. As a result, the methane production inhibitor may further improve production efficiency of the volatile organic acids. Here, CHI₃ or CHBr₃ may be used as the methane production inhibitor. In particular, according to Example embodiments of the present invention, it is confirmed that production of the VFAs is the most effective when CHI₃ is used as the methane production inhibitor.

The anaerobic fermentation of Step 4) plays an important role in regulating pH and osmotic pressure, and may be performed in a medium including a microorganism and an inorganic salt. The inorganic salt may be at least one selected from the group consisting of inorganic salts such as ammonium salt, phosphate, calcium salt, magnesium salt and sodium salt. In particular, the medium may include (NH₂)₂CO as a nitrogen source and KH₂PO₄ as a phosphorus source. According to Example embodiments of the present invention, it is confirmed that it is most effective to produce the VFAs in a medium including (NH₂)₂CO and KH₂PO₄ so that nitrogen and phosphorus can be present at a molar ratio of 1.5:1 to 7.5:1.

The VFAs produced through the anaerobic fermentation may include, but are not limited to, propionic acid, butyric acid, valeric acid or caproic acid, as well as acetic acid (i.e., both of the iso- and normal formation). In particular, kinds of the produced VFAs may vary according to the conditions such as fermentation acidity (pH), fermentation temperature, kinds of algae, etc. A person skilled in the art can selectively produce a desired VFA by properly adjusting the conditions.

Hereinafter, Example embodiments of the present invention will be described in detail with reference to the following Examples.

However, it should be understood that the description proposed herein is merely a preferable example for the purpose of illustration only, not intended to limit the scope of the invention.

### Example 1

### Preparation of Pretreated Extract of Algae for Production of VFAs

<1-1> Collection of Residue Remaining after Recovery of Crude Ethanol Extract from Algae

Each of a brown alga, Laminaria japonica (100 kg), a red alga, Pachymeniopsis lanceolata (25 kg), and a green alga, Enteromorpha crinita (25 kg), was naturally dried, and ground into powder having a diameter of 10 mm or less using a grinding mill. 15 kg of the physically pretreated algae powder and 75 kg of ethanol were mixed, and the resulting mixture was repeatedly extracted in triplicate at 80° C. at regular intervals under cold reflux, and filtered through a filter paper under a reduced pressure. Thereafter, the filtered dreg (i.e., a residue) was used as a sample to produce VFAs.

<1-2> Pretreatment of Algae Residue

The residue of algae obtained in Example <1-1> was chemically or biologically pretreated using methods listed in the following Table 1, and the alga pretreated as described above was filtered to obtain a filtrate. Concentrations of the extract filtrates are listed in the following Table 2. Here, the concentrations listed in the following Table 2 were calculated according to the following Equation 1. A concentration of the extract filtrate was adjusted to a desired level by diluting or concentrating the extract filtrate.

TABLE 1

| Pretreatment | | |
|---|---|---|
| type | Pretreatment method | Condition |
| Biological | Microorganism | Kind of microorganism: abalone viscera-derived |
| pretreatment | treatment method | microorganism |
| | | Kind of culture broth: LB broth (tryptone, yeast extract, |
| | | sodium chloride) |
| | | Culture temperature and time: 37° C., 36 hours |
| Chemical | First acid catalyst | Reaction system: batch-type autoclave reactor |
| pretreatment | treatment method | Catalyst: An aqueous solution containing 3 wt % H₂SO₄ |
| | | Reaction time: 250 minutes |
| | Second acid catalyst | Reaction system: reflux-type autoclave reactor |
| | treatment method | Catalyst: An aqueous solution containing 5 wt % H₂SO₄ |
| | | Reaction time: 500 minutes |
| | Base catalyst treatment | Reaction system: batch-type autoclave reactor |
| | method | Catalyst: An aqueous solution containing 1 wt % NaOH |
| | | Reaction temperature: 120° C. |
| | | Reaction time: 60 minutes |
| | Supercritical water | <Step 1: Treatment with supercritical carbon dioxide> |
| | treatment method after | Reaction system: continuous autoclave extraction |
| | treatment with | apparatus |
| | supercritical carbon | Catalyst and solvent: carbon dioxide |
| | dioxide | Reaction temperature and pressure: 45° C., 200 bars |
| | | Reaction time: 1 hour |
| | | <Step 2: Treatment with supercritical carbon dioxide> |
| | | Reaction system: batch-type autoclave reactor |
| | | Catalyst and solvent: water |
| | | Reaction temperature and pressure: 300° C., 250 bars |
| | | Reaction time: 1 minute |
| | Treatment with | Reaction system: batch-type autoclave reactor |
| | supercritical water | Catalyst and solvent: water |
| | | Reaction temperature and pressure: 300° C., 250 bars |
| | | Reaction time: 1 minute |

TABLE 2

| | | Concentration of |
|---|---|---|
| Pretreatment method | Kind of algae | extract filtrate (g/L) |
| Biological pretreatment | Laminaria japonica | 6.0 |
| First acid catalyst treatment method | Laminaria japonica | 84.0 |
| | Pachymeniopsis lanceolata | 84.5 |
| | Enteromorpha crinita | 51.2 |
| Second acid catalyst treatment method | Laminaria japonica | 79.5 |
| Base catalyst treatment method | Laminaria japonica | 12.1 |
| | Pachymeniopsis lanceolata | 12.0 |
| | Enteromorpha crinita | 11.9 |
| Supercritical water treatment method after | Laminaria japonica | 78.3 |
| treatment with supercritical carbon dioxide | | |
| Supercritical water treatment method | Laminaria japonica | 78.7 |

$$\begin{matrix}Concentration of \\ extract ( g \text{/} L )\end{matrix} = \frac{\begin{matrix}Total weight ( g ) of dried algae before extraction - \\ Total weight ( g ) of dried algae after extraction\end{matrix}}{Volume ( L ) of solvent} \qquad Equation 1$$

<1-3> Production of VFAs

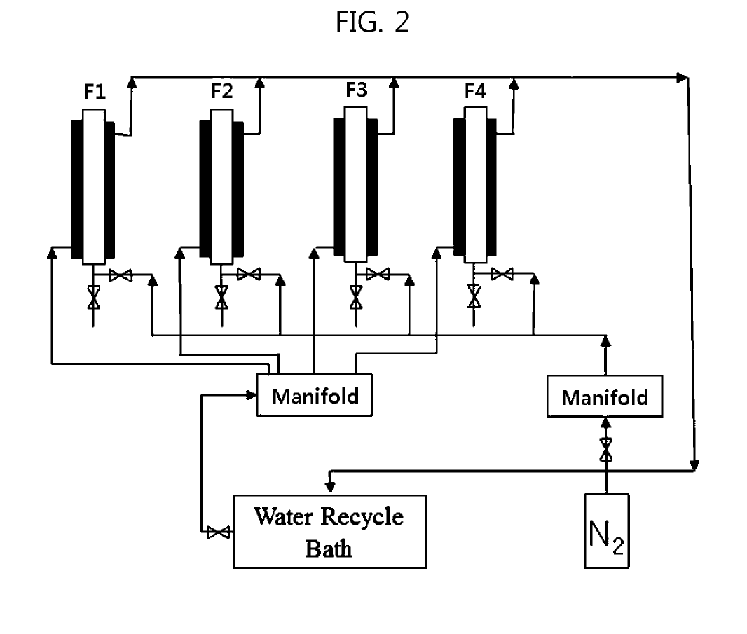

The filtrate obtained in Example <1-2> was diluted, and a medium having a composition listed in the following Table 3 and a methane production inhibitor were put into a fermentation apparatus shown in FIG. 2 together with a microorganism (at a volume ratio of 1/15, that is, 0.06 L of a microorganism based on a 0.9 L of a fermentation solution) (obtained from an anaerobic fermentation tank in a sewage disposal plant at SuYoung, Busan). Thereafter, nitrogen gas was added for 10 minutes so as to remove oxygen gas in the fermentation apparatus. In order to adjust a pH value changed by the presence of a fermentation product during incubation, the pH value was maintained to a desired constant level by adding a 3 M NH₄HCO₃ solution when the pH value decreased and a 3 M H₂PO₄ solution when the pH value increased. Also, a fermentation temperature in the fermentation apparatus was maintained to a desired constant level using a thermostat. Nitrogen gas was added from the bottom of the fermentation apparatus to mix the components in the fermentation apparatus, and the mixing was performed twice a day. The yield of VFAs from the collected samples was quantified using a gas chromatography with flame ionization detector (Shimadzu 17A), and calculated according to the following Equation 2.

TABLE 3

| | Medium compositions (content) | N:P molar ratio |
|---|---|---|
| Medium 1 | NH₄HCO₃ (2.00 g), KH₂PO₄ (1.00 g), MgSO₄•7H₂O (0.01 g), | 35:1 |
| | NaCl (0.001 g), Na₂MoO₄•2H₂O (0.001 g), CaCl₂•2H₂O (0.001 g), | |
| | MnSO₄•7H₂O (0.0015 g), FeCl₂ (0.00278 g) | |
| Medium 2 | (NH₂)₂CO (0.76 g), KH₂PO₄ (1.00 g) | 35:1 |
| Medium 3 | NH₄HCO₃ (2.00 g), KH₂PO₄ (1.00 g) | 35:1 |
| Medium 4 | NH₄Cl (1.35 g), Na₂HPO₄•2H₂O (1.31 g) | 35:1 |
| Medium 5 | NH₄Cl (1.35 g), Na₂HPO₄•2H₂O (0.99 g), KH₂PO₄ (0.25 g) | 35:1 |

$$Yield ( \% ) = \frac{\begin{matrix}Concentration ( g \text{/} L ) of introduced extract - \\ Concentration ( g \text{/} L ) of generated volatile fatty acids\end{matrix}}{Concentration ( g \text{/} L ) of introduced extract} \qquad Equation 2$$

### Example 2

### Analysis of Concentration and Yield of VFAs According to Conditions

VFAs were produced from a residue of an algae extract in the same manner as in Example 1, except that the conditions such as a pretreatment method, kinds of algae, a concentration of an extract, a volume ratio of a microorganism, a fermentation temperature and fermentation pH were changed to analyze the concentration and yield of the VFAs.

<2-1> Analysis of Concentration and Yield of VFAs According to Pretreatment Method

In order to analyze the concentration and yield of VFAs according to the pretreatment method, the VFAs were produced while constantly maintaining all the other conditions listed in the following Tables 4 to 6, except for the pretreatment method performed before anaerobic fermentation. First, the algae extracts pretreated using a first acid catalyst treatment method, a base catalyst treatment method and a supercritical water treatment method were compared with each other. As a result, it could be seen that the highest concentration and yield of the VFAs were produced from the residue of the algae extract pretreated using the first acid catalyst treatment method and the base catalyst treatment method, as listed in Table 4.

TABLE 4

| | | | | | | | Day 5 | | Day 10 | | Day 15 | | Day 20 | |
|---|---|---|---|---|---|---|---|---|---|---|---|---|---|---|
| | | Concen- | Volume | Fermen- | | | Concen- | | Concen- | | Concen- | | Concen- | |
| | | tration | ratio | tation | | | tration | | tration | | tration | | tration | |
| Pretreat- | Kinds | (g/L) | of | temper- | | Kinds | (g/L) of | | (g/L) of | | (g/L) of | | (g/L) of | |
| ment | of | of | micro- | ature | | of | organic | Yield | organic | Yield | organic | Yield | organic | Yield |
| method | algae | extract | organism | (° C.) | pH | media | acid | (%) | acid | (%) | acid | (%) | acid | (%) |
| First acid | Laminaria | 54 | 1/15 | 35 | 7 | Medium 1 | 2.6 | 4.8 | 17.6 | 32.6 | 20.0 | 37.0 | 21.4 | 39.6 |
| catalyst | japonica | | | | | | | | | | | | | |
| treatment | | | | | | | | | | | | | | |
| method | | | | | | | | | | | | | | |
| Second acid | Laminaria | | | | | | 8.4 | 15.5 | 20.5 | 38.0 | 21.7 | 40.2 | 19.2 | 35.6 |
| catalyst | japonica | | | | | | | | | | | | | |
| treatment | | | | | | | | | | | | | | |
| method | | | | | | | | | | | | | | |
| Supercritical | Laminaria | | | | | | 1.7 | 3.1 | 3.1 | 5.7 | 4.3 | 8.0 | 4.7 | 8.7 |
| water | japonica | | | | | | | | | | | | | |
| treatment | | | | | | | | | | | | | | |
| method | | | | | | | | | | | | | | |

Next, the algae extracts pretreated using a first acid catalyst treatment method and a microorganism treatment method were compared with each other. When Laminaria japonica was pretreated using a method such as treatment with a first acid catalyst, a concentration of the Laminaria japonica extract was 54 g/L as listed in Table 4, which was relatively higher than a concentration (6 g/L) of the extract obtained when Laminaria japonica was pretreated using the microorganism treatment method. However, the Laminaria japonica extract was properly diluted to a concentration of 6 g/L for comparison with the microorganism treatment method.

As a result, the VFAs were produced with relatively higher concentration and yield in the microorganism treatment method than in the first acid catalyst treatment method, as listed in Table 5. From the described-above results, it could be seen that, when the extract was obtained from the algae residue using the microorganism treatment method, a large amount of the components from which the VFAs were produced was selectively included in the extract.

TABLE 5

| | | | | | | | Day 5 | | Day 10 | | Day 15 | | Day 20 | |
|---|---|---|---|---|---|---|---|---|---|---|---|---|---|---|
| | | Concen- | Volume | Fermen- | | | Concen- | | Concen- | | Concen- | | Concen- | |
| | | tration | ratio | tation | | | tration | | tration | | tration | | tration | |
| Pretreat- | Kinds | (g/L) | of | temper- | | Kinds | (g/L) of | | (g/L) of | | (g/L) of | | (g/L) of | |
| ment | of | of | micro- | ature | | of | organic | Yield | organic | Yield | organic | Yield | organic | Yield |
| method | algae | extract | organism | (° C.) | pH | media | acid | (%) | acid | (%) | acid | (%) | acid | (%) |
| Microorganism | Laminaria | 6 | 1/15 | 35 | 7 | Medium 1 | 4.4 | 73.3 | 5.3 | 88.3 | 5.7 | 95.0 | 5.6 | 93.3 |
| treatment | japonica | | | | | | | | | | | | | |
| method | | | | | | | | | | | | | | |
| First acid | Laminaria | 6 | | | | | 0.9 | 15.0 | 2.0 | 33.3 | 2.5 | 41.7 | 2.6 | 43.3 |
| catalyst | japonica | | | | | | | | | | | | | |
| treatment | | | | | | | | | | | | | | |
| method | | | | | | | | | | | | | | |

Finally, the algae extracts pretreated using a first acid catalyst treatment method, a second acid catalyst treatment method and a supercritical water treatment method were compared with each other. As a result, it could be seen that the VFAs were produced with the highest concentration and yield from the algae extract pretreated using the first acid catalyst treatment method, as listed in Table 6. The second acid catalyst treatment method might be advantageous for mass production due to low apparatus costs since it does not require a high-pressure reactor, compared with the first acid catalyst treatment method.

TABLE 6

| | | | | | | | Day 5 | | Day 10 | | Day 15 | | Day 20 | |
|---|---|---|---|---|---|---|---|---|---|---|---|---|---|---|
| | | Concen- | Volume | Fermen- | | | Concen- | | Concen- | | Concen- | | Concen- | |
| | | tration | ratio | tation | | | tration | | tration | | tration | | tration | |
| Pretreat- | Kinds | (g/L) | of | temper- | | Kinds | (g/L) of | | (g/L) of | | (g/L) of | | (g/L) of | |
| ment | of | of | micro- | ature | | of | organic | Yield | organic | Yield | organic | Yield | organic | Yield |
| method | algae | extract | organism | (° C.) | pH | media | acid | (%) | acid | (%) | acid | (%) | acid | (%) |
| First acid | Laminaria | 18 | 1/15 | 35 | 7 | Medium 1 | 2.88 | 14.4 | 6.76 | 33.8 | 7.64 | 38.2 | 8.95 | 44.8 |
| catalyst | japonica | | | | | | | | | | | | | |
| treatment | | | | | | | | | | | | | | |
| method | | | | | | | | | | | | | | |
| Second acid | Laminaria | | | | | | 1.58 | 7.90 | 3.50 | 17.50 | 4.08 | 20.4 | 3.25 | 16.25 |
| catalyst | japonica | | | | | | | | | | | | | |
| treatment | | | | | | | | | | | | | | |
| method | | | | | | | | | | | | | | |
| Supercritical | Laminaria | | | | | | 4.86 | 26.0 | 5.18 | 25.9 | 3.52 | 17.6 | 2.80 | 14.0 |
| water | japonica | | | | | | | | | | | | | |
| treatment | | | | | | | | | | | | | | |
| method | | | | | | | | | | | | | | |

From the described-above results, it could be seen that the VFAs were produced with high concentration and yield from the algae extracts pretreated using the first acid catalyst treatment method and the base treatment method. Among these, it could be seen that the first acid catalyst treatment method was most effective. Therefore, in the following experiments using the other conditions, the algae were pretreated using the first acid catalyst treatment method, and the concentration and yield of the VFAs were compared and analyzed.

<2-2> Analysis of Concentration and Yield of VFA According to Kinds of Algae

In order to analyze the concentration and yield of VFAs according to the kinds of algae, the VFAs were produced while constantly maintaining all the other conditions listed in the following Table 7, except that the different kinds of the algae, for example, Laminaria japonica, Pachymeniopsis lanceolata, Enteromorpha crinita and Chlorella, were used, and the different pretreatment methods, for examples, a first acid catalyst treatment method, a second acid catalyst treatment method and a base catalyst treatment method, were used herein.

TABLE 7

| | | | | | | | Day 5 | | Day 10 | | Day 15 | | Day 20 | |
|---|---|---|---|---|---|---|---|---|---|---|---|---|---|---|
| | | Concen- | Volume | Fermen- | | | Concen- | | Concen- | | Concen- | | Concen- | |
| | | tration | ratio | tation | | | tration | | tration | | tration | | tration | |
| Pretreat- | Kinds | (g/L) | of | temper- | | Kinds | (g/L) of | | (g/L) of | | (g/L) of | | (g/L) of | |
| ment | of | of | micro- | ature | | of | organic | Yield | organic | Yield | organic | Yield | organic | Yield |
| method | algae | extract | organism | (° C.) | pH | media | acid | (%) | acid | (%) | acid | (%) | acid | (%) |
| First acid | Laminaria | 18 | 1/15 | 35 | 7 | Medium 1 | 2.88 | 14.4 | 6.76 | 33.8 | 7.64 | 38.2 | 8.95 | 44.8 |
| catalyst | japonica | | | | | | | | | | | | | |
| treatment | Pachymeniopsis | | | | | | 4.9 | 24.3 | 6.64 | 33.2 | 6.02 | 30.1 | 5.56 | 27.8 |
| method | lanceolata | | | | | | | | | | | | | |
| | Enteromorpha | | | | | | 4.2 | 20.9 | 6.45 | 32.3 | 5.31 | 26.6 | 4.97 | 24.9 |
| | crinita | | | | | | | | | | | | | |
| Second acid | Chlorella | 18 | 1/15 | 35 | 7 | Medium 1 | 4.2 | 23.3 | 4.9 | 27.2 | 6.1 | 33.9 | 5.6 | 3.11 |
| catalyst | | | | | | | | | | | | | | |
| treatment | | | | | | | | | | | | | | |
| method | | | | | | | | | | | | | | |
| Base catalyst | Laminaria | 54 | 1/15 | 35 | 7 | Medium 1 | 18.4 | 34.1 | 20.5 | 38.0 | 21.7 | 40.2 | 19.2 | 35.6 |
| treatment | japonica | | | | | | | | | | | | | |
| method | Pachymeniopsis | | | | | | 17.2 | 31.9 | 17.2 | 31.9 | 15.2 | 28.1 | 16.0 | 29.6 |
| | lanceolata | | | | | | | | | | | | | |
| | Enteromorpha | | | | | | 19.6 | 36.3 | 21.9 | 40.6 | 21.0 | 38.9 | 16.9 | 31.3 |
| | crinita | | | | | | | | | | | | | |

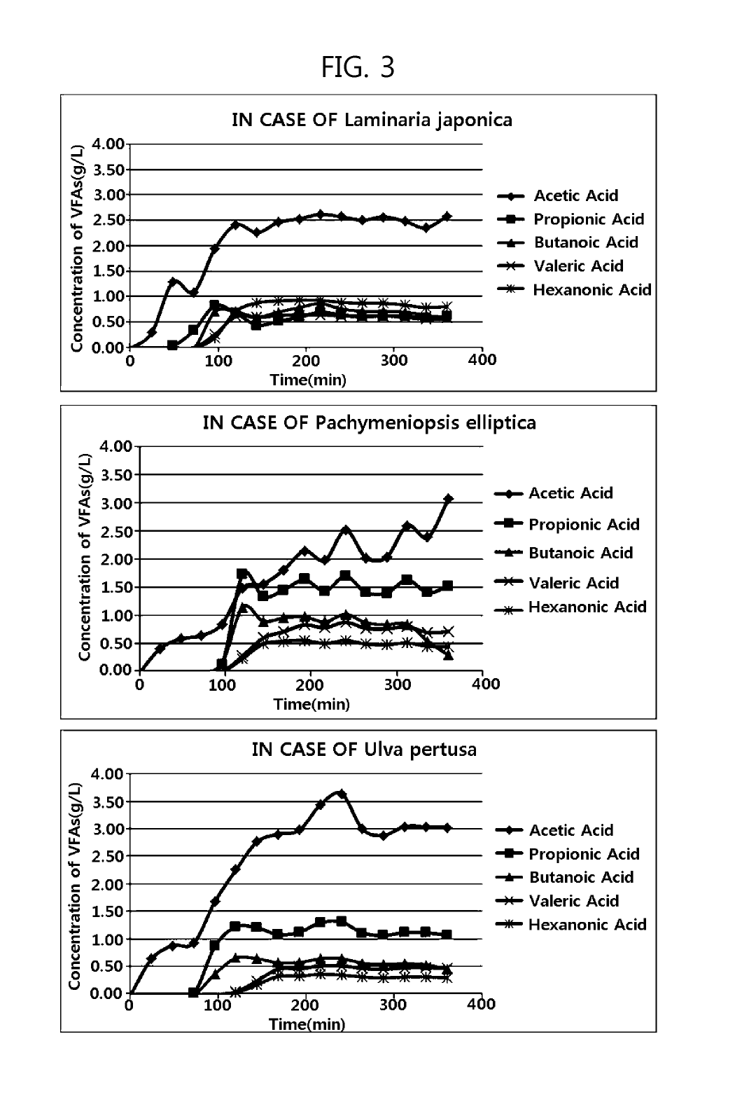

As a result, the VFAs were produced with high concentration and yield from all kinds of the algae regardless of the pretreatment method, as listed in Table 7. Among these, however, the compositions of the produced VFAs varied according to the kinds of the algae, as shown in FIG. 3. Also, a relatively high concentration of acetic acid was produced from the brown algae, Laminaria japonica, and the green algae, Enteromorpha crinita, and acetic acid, propionic acid and butyric acid were produced in order of increasing concentration from the red algae, Pachymeniopsis lanceolata.

<2-3> Analysis of Concentration and Yield of VFA According to Pretreatment Conditions

In order to analyze the concentration and yield of VFAs according to the concentrations of the pretreated extracts, the VFAs were produced while constantly maintaining all the other conditions listed in the following Table 8, except that the pretreatment conditions for the first acid catalyst treatment method, for example, a reaction temperature, an acid catalyst concentration and a reaction time, were changed.

TABLE 8

| Pretreatment condition | | | | | | | | | | |
|---|---|---|---|---|---|---|---|---|---|---|
| | | Concen- | | | | Concen- | Volume | Fermen- | | |
| | Reaction | tration | | | | tration | ratio | tation | | |
| | temper- | (wt %) of | Reaction | Pretreat- | Kinds | (g/L) | of | temper- | | Kinds |
| | ature | acid | time | ment | of | of | micro- | ature | | of |
| No. | (° C.) | catalyst | (min) | method | algae | extract | organism | (° C.) | pH | media |
| 1 | 100 | 1 | 100 | First | Laminaria | 18 | 1/15 | 35 | 7 | Medium 1 |
| 2 | | | 400 | acid | japonica | | | | | |
| 3 | | 5 | 100 | catalyst | | | | | | |
| 4 | | | 400 | treatment | | | | | | |
| 5 | | 10 | 100 | method | | | | | | |
| 6 | | | 400 | | | | | | | |
| 7 | 120 | 1 | 100 | | Laminaria | 18 | 1/15 | 35 | 7 | Medium 1 |
| 8 | | | 400 | | japonica | | | | | |
| 9 | | 5 | 100 | | | | | | | |
| 10 | | | 400 | | | | | | | |
| 11 | | 10 | 100 | | | | | | | |
| 12 | | | 400 | | | | | | | |

| Pretreatment condition | | | | Day 5 | | Day 10 | | Day 15 | |
|---|---|---|---|---|---|---|---|---|---|
| | | Concen- | | Concen- | | Concen- | | Concen- | |
| | Reaction | tration | | tration | | tration | | tration | |
| | temper- | (wt %) of | Reaction | (g/L) of | | (g/L) of | | (g/L) of | |
| | ature | acid | time | organic | Yield | organic | Yield | organic | Yield |
| No. | (° C.) | catalyst | (min) | acid | (%) | acid | (%) | acid | (%) |
| 1 | 100 | 1 | 100 | 6.2 | 34.4 | 5.9 | 32.8 | 5.4 | 30.0 |
| 2 | | | 400 | 6.5 | 36.1 | 5.6 | 31.1 | 5.1 | 28.3 |
| 3 | | 5 | 100 | 7.2 | 40.0 | 6.3 | 35.0 | 5.4 | 30.0 |
| 4 | | | 400 | 5.7 | 31.7 | 4.8 | 26.7 | 4.4 | 24.4 |
| 5 | | 10 | 100 | 5.9 | 32.8 | 6.3 | 35.0 | 4.9 | 37.2 |
| 6 | | | 400 | 5.4 | 30.0 | 5.4 | 30.0 | 4.6 | 25.6 |
| 7 | 120 | 1 | 100 | 6.1 | 33.9 | 4.8 | 26.7 | 5.0 | 27.8 |
| 8 | | | 400 | 3.7 | 20.6 | 4.1 | 22.8 | 4.7 | 26.1 |
| 9 | | 5 | 100 | 5.8 | 32.2 | 5.0 | 27.8 | 5.2 | 28.9 |
| 10 | | | 400 | 3.7 | 20.6 | 3.4 | 18.9 | 3.6 | 20.0 |
| 11 | | 10 | 100 | 7.9 | 43.9 | 6.4 | 35.6 | 5.7 | 31.7 |
| 12 | | | 400 | 2.9 | 16.1 | 3.3 | 18.3 | 3.2 | 17.8 |

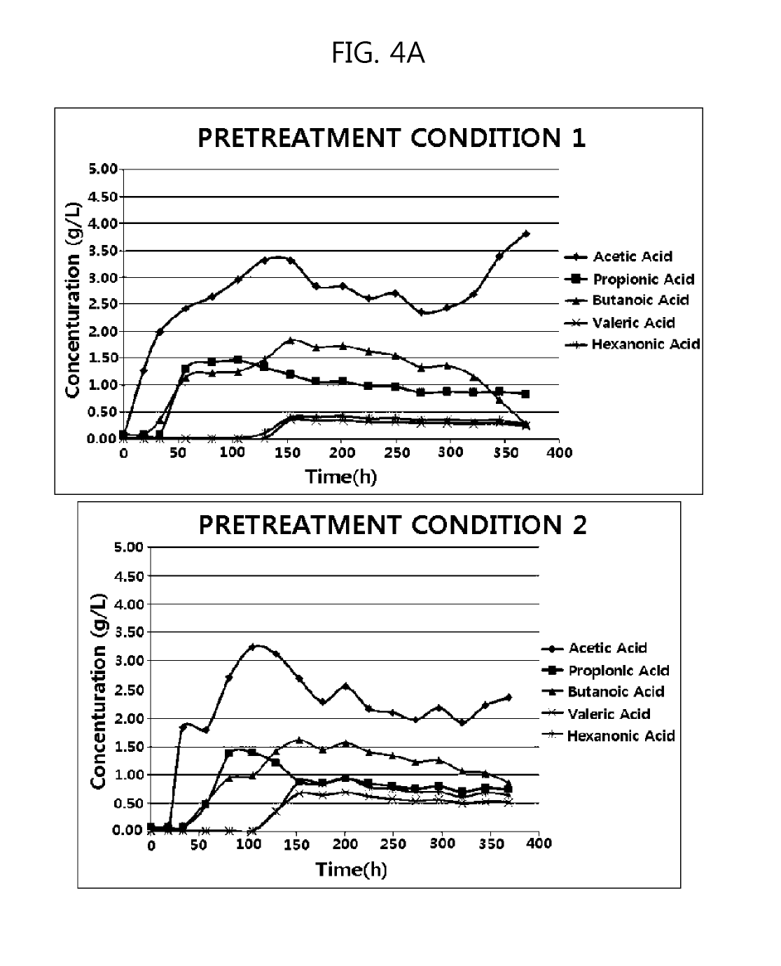

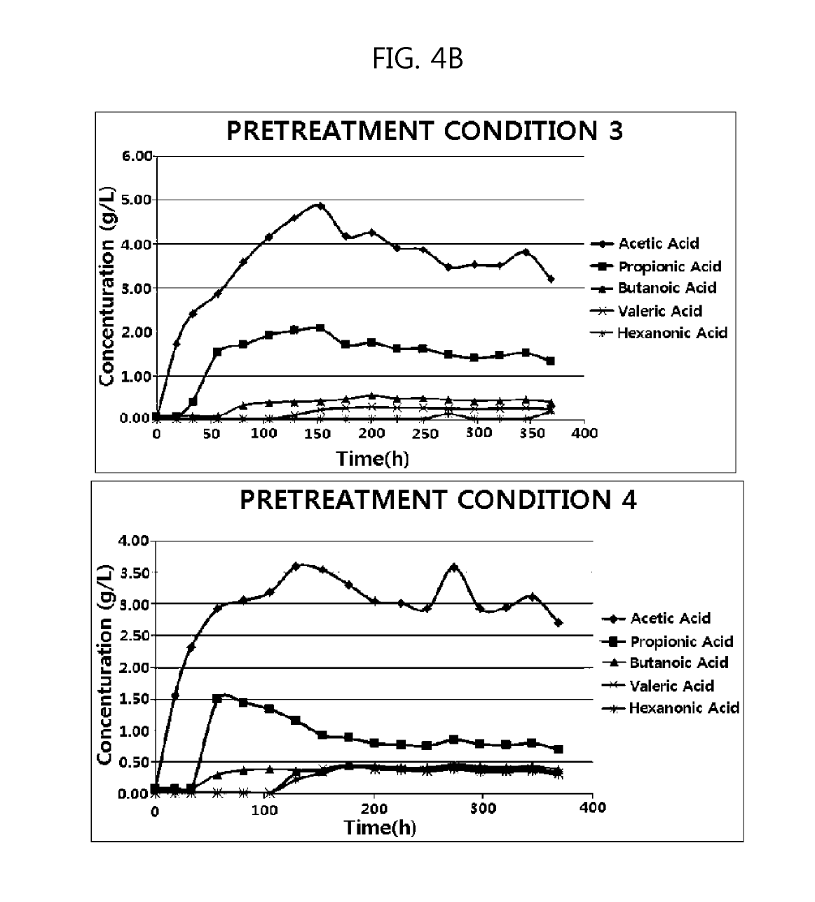

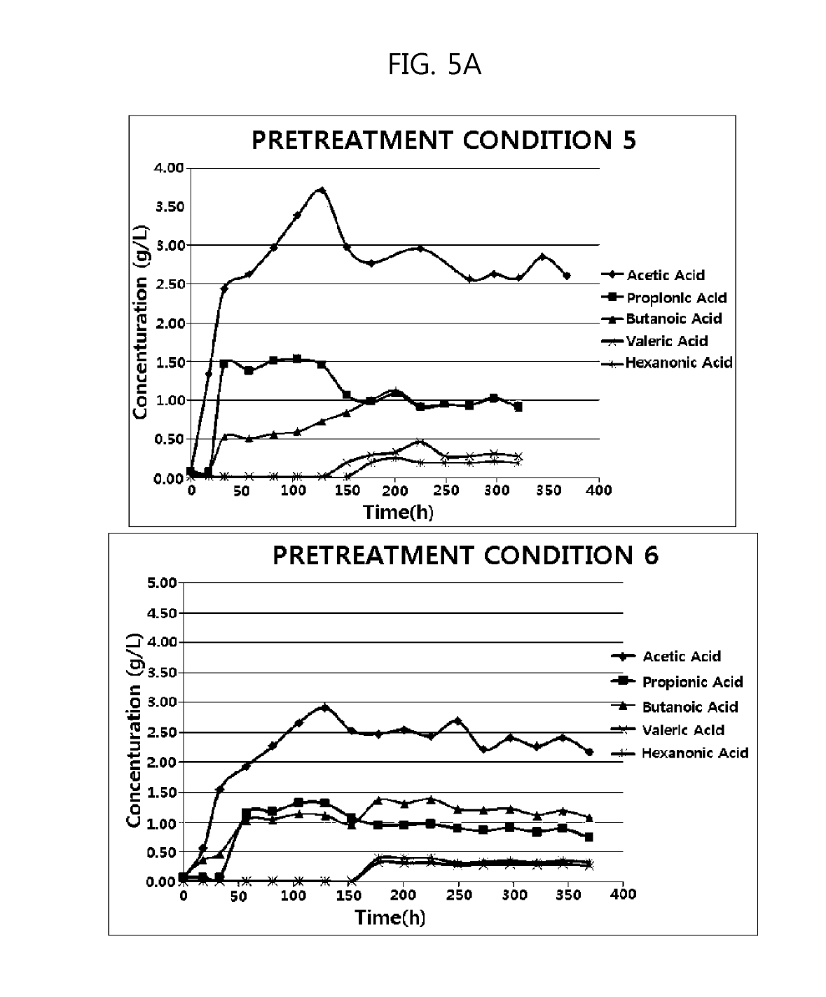

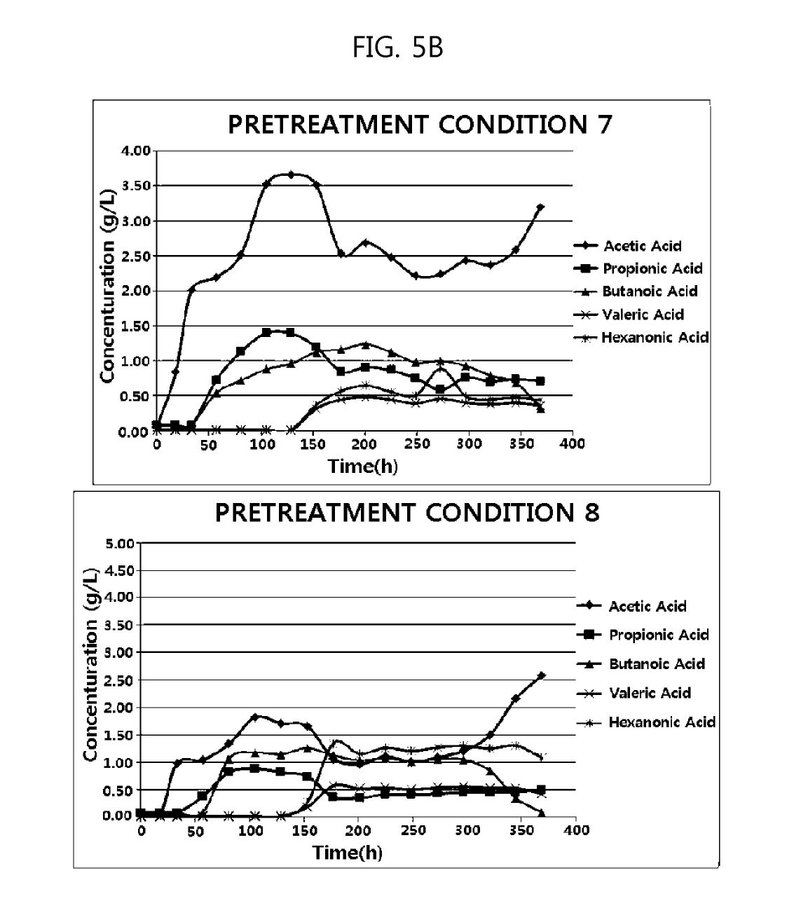

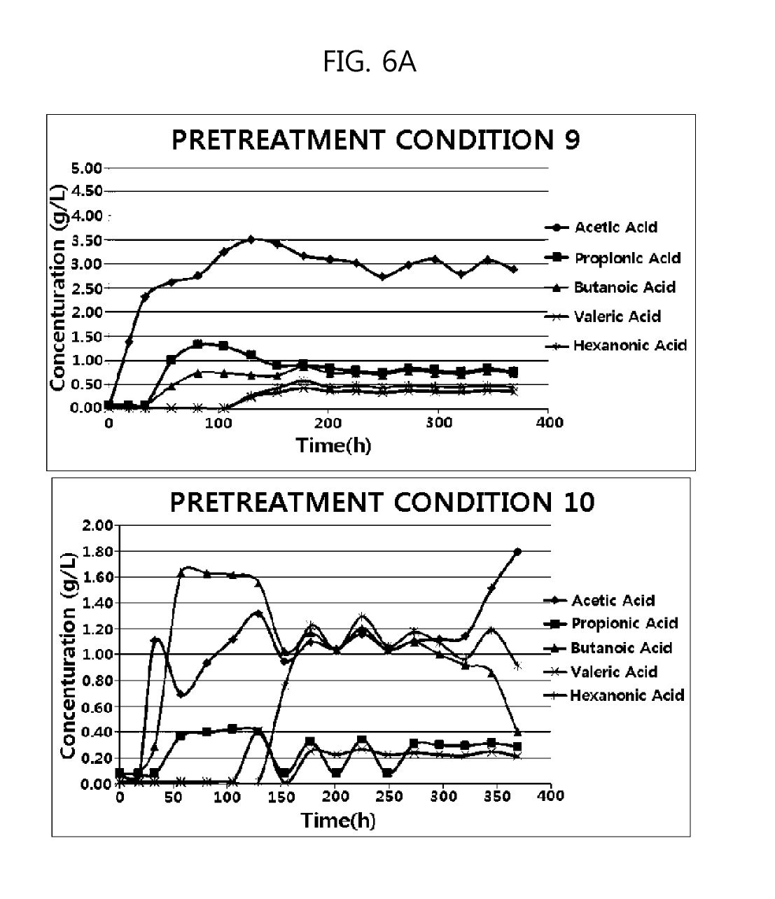

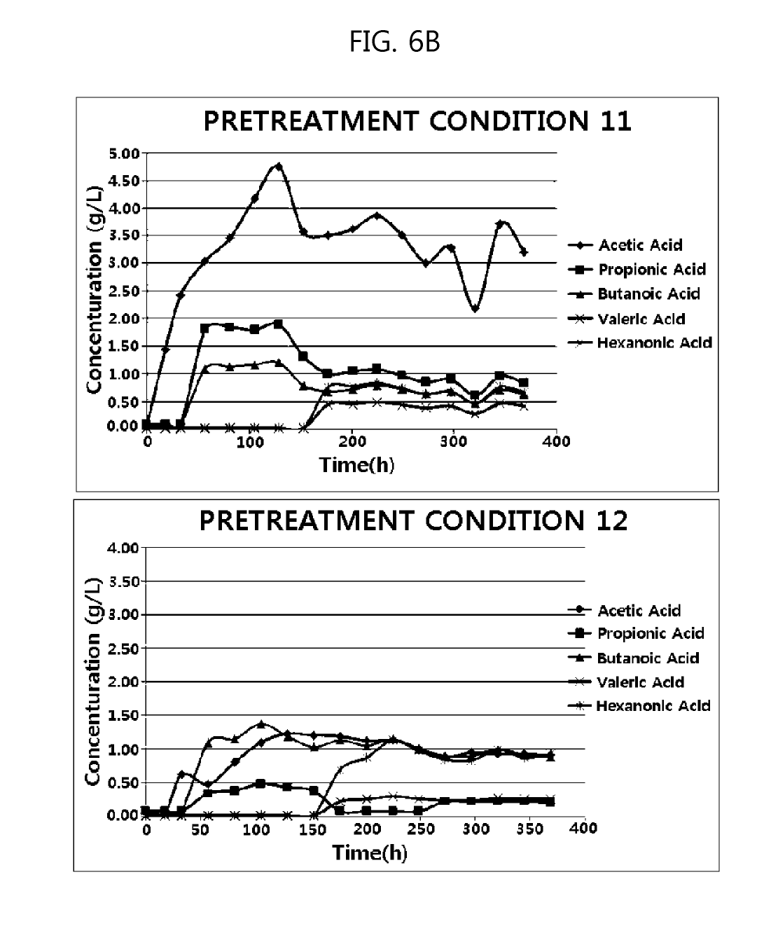

As a result, the VFAs were readily produced under the pretreatment conditions such as a reaction temperature of 100° C. to 120° C., an acid catalyst concentration of 1 wt % to 10 wt % and a reaction time of 100 to 400 minutes, and reached the maximum concentration 5 days after the anaerobic fermentation, as listed in Table 8. Also, the compositions of the produced VFAs were different according to the pretreatment conditions, as shown in FIG. 4 to FIG. 6. In general, acetic acid, propionic acid and butyric acid were produced in order of increasing concentration, and butyric acid, caproic acid and valeric acid were produced in an increasing concentration as the reaction time progressed.

<2-4> Analysis of Concentration and Yield of VFA According to Concentrations of Pretreated Extracts

In order to analyze the concentration and yield of VFAs according to concentrations of the pretreated extracts, the VFAs were produced while constantly maintaining all the other conditions listed in the following Table 9, except that the pretreated algae extract was used at different concentrations of 18 g/L, 36 g/L, 54 g/L and 72 g/L.

TABLE 9

| | | | | | | | Day 5 | | Day 10 | | Day 15 | | Day 20 | |
|---|---|---|---|---|---|---|---|---|---|---|---|---|---|---|
| | | Concen- | Volume | Fermen- | | | Concen- | | Concen- | | Concen- | | Concen- | |
| | | tration | ratio | tation | | | tration | | tration | | tration | | tration | |
| Pretreat- | Kinds | (g/L) | of | temper- | | Kinds | (g/L) of | | (g/L) of | | (g/L) of | | (g/L) of | |
| ment | of | of | micro- | ature | | of | organic | Yield | organic | Yield | organic | Yield | organic | Yield |
| method | algae | extract | organism | (° C.) | pH | media | acid | (%) | acid | (%) | acid | (%) | acid | (%) |
| First | Laminaria | 20 | 1/15 | 35 | 7 | CHI₃ | 2.88 | 14.4 | 6.76 | 33.8 | 7.64 | 38.2 | 8.95 | 44.8 |
| acid | japonica | 40 | | | | | 3.8 | 9.6 | 12.3 | 30.9 | 14.25 | 35.6 | 15.85 | 39.6 |
| catalyst | | 60 | | | | | 2.6 | 4.3 | 17.6 | 29.4 | 20.0 | 33.3 | 21.4 | 35.6 |
| treatment | | 80 | | | | | 11.6 | 14.5 | 18.09 | 22.6 | 22.3 | 27.9 | 18.83 | 23.5 |
| method | | | | | | | | | | | | | | |

As a result, a concentration of the VFAs was increased with an increasing concentration of the extract of the pretreated algae residue, and the concentration of the VFAs was not increased when the extract was present at a concentration of 54 g/L or more, as listed in Table 9. This indicates that a microorganism reached its limit to produce the VFAs, and thus the concentration of the VFAs was not increased any more, and the yield of the VFAs was decreased due to an increase of an unreacted extract.

<2-5> Analysis of Concentration and Yield of VFA According to Volume Ratio of Anaerobic Fermentation Microorganism

In order to analyze the concentration and yield of VFAs according to a volume ratio of an anaerobic fermentation microorganism, the VFAs were produced while constantly maintaining all the other conditions listed in the following Table 10, except that an extract of the Laminaria japonica residue pretreated using a first acid catalyst treatment method, a supercritical water treatment method after treatment with supercritical carbon dioxide and a supercritical water treatment method was treated with an anaerobic fermentation microorganism at volume ratios of 1/30, 1/15 and 1/10, based on the total amount of the filtrated to be fermented, to produce VFAs.

TABLE 10

| | | | | | | | Day 5 | | Day 10 | | Day 15 | | Day 20 | |
|---|---|---|---|---|---|---|---|---|---|---|---|---|---|---|
| | | Concen- | Volume | Fermen- | | | Concen- | | Concen- | | Concen- | | Concen- | |
| | | tration | ratio | tation | | | tration | | tration | | tration | | tration | |
| Pretreat- | Kinds | (g/L) | of | temper- | | Kinds | (g/L) of | | (g/L) of | | (g/L) of | | (g/L) of | |
| ment | of | of | micro- | ature | | of | organic | Yield | organic | Yield | organic | Yield | organic | Yield |
| method | algae | extract | organism | (° C.) | pH | media | acid | (%) | acid | (%) | acid | (%) | acid | (%) |
| First acid | Laminaria | 18 | 1/30 | 45 | 7 | Medium 1 | 3.2 | 17.8 | 5.2 | 28.9 | 5.6 | 31.1 | 6.0 | 33.3 |
| catalyst | japonica | | 1/15 | | | | 4.5 | 25.0 | 6.0 | 33.3 | 6.6 | 36.7 | 6.2 | 34.4 |
| treatment | | | 1/10 | | | | 4.7 | 26.1 | 6.4 | 35.6 | 6.2 | 34.4 | 6.3 | 35.0 |
| method | | | | | | | | | | | | | | |
| Second acid | Laminaria | 18 | 1/30 | 35 | 7 | Medium 1 | 3.3 | 18.3 | 4.7 | 26.1 | 3.5 | 19.4 | 3.0 | 16.7 |
| catalyst | japonica | | 1/15 | | | | 4.9 | 27.2 | 5.2 | 28.9 | 3.5 | 19.4 | 2.8 | 15.6 |
| treatment | | | 1/10 | | | | 5.2 | 28.9 | 4.7 | 26.1 | 4.0 | 22.2 | 3.5 | 19.4 |
| method | | | | | | | | | | | | | | |
| Supercritical | Laminaria | 18 | 1/30 | 35 | 7 | Medium 1 | 1.7 | 9.4 | 3.1 | 17.2 | 3.1 | 17.2 | 2.0 | 11.1 |
| water | japonica | | 1/15 | | | | 2.1 | 11.7 | 2.8 | 15.6 | 3.6 | 20.0 | 1.5 | 8.3 |
| treatment | | | 1/10 | | | | 2.6 | 14.4 | 2.8 | 1.56 | 3.3 | 18.3 | 2.6 | 14.4 |
| method | | | | | | | | | | | | | | |

As a result, the concentration and yield of the VFAs increased with an increasing volume ratio of the microorganism regardless of the pretreatment method, and the microorganism was used at the optimum volume ratio of 1/15, as listed in Table 10. When the microorganism was used at a volume ratio of 1/15 or more, a concentration of the produced VFAs was not increased any more, and an amount of microorganism sludge increased, which leads to an increase in separation costs.

<2-6> Analysis of Concentration and Yield of VFA According to Fermentation Temperature

In order to analyze the concentration and yield of VFA according to a fermentation temperature, the VFAs were produced while constantly maintaining all the other conditions listed in the following Table 11, except for different fermentation temperatures of 25° C., 35° C. and 45° C.

TABLE 11

| | | | | | | | Day 5 | | Day 10 | | Day 15 | | Day 20 | |
|---|---|---|---|---|---|---|---|---|---|---|---|---|---|---|
| | | Concen- | Volume | Fermen- | | | Concen- | | Concen- | | Concen- | | Concen- | |
| | | tration | ratio | tation | | | tration | | tration | | tration | | tration | |
| Pretreat- | Kinds | (g/L) | of | temper- | | Kinds | (g/L) of | | (g/L) of | | (g/L) of | | (g/L) of | |
| ment | of | of | micro- | ature | | of | organic | Yield | organic | Yield | organic | Yield | organic | Yield |
| method | algae | extract | organism | (° C.) | pH | media | acid | (%) | acid | (%) | acid | (%) | acid | (%) |
| First acid | Laminaria | 18 | 1/15 | 25 | 7 | Medium 1 | 4.0 | 22.2 | 6.4 | 35.6 | 6.4 | 35.6 | 6.6 | 36.7 |
| catalyst | japonica | | | | | | | | | | | | | |
| treatment | Laminaria | | | | | | 2.9 | 16.1 | 6.8 | 37.8 | 7.6 | 42.2 | 9.0 | 50.0 |
| method | japonica | | | | | | | | | | | | | |
| | Laminaria | | | | | | 4.5 | 25.0 | 6.0 | 33.3 | 6.6 | 36.7 | 6.2 | 34.4 |
| | japonica | | | | | | | | | | | | | |

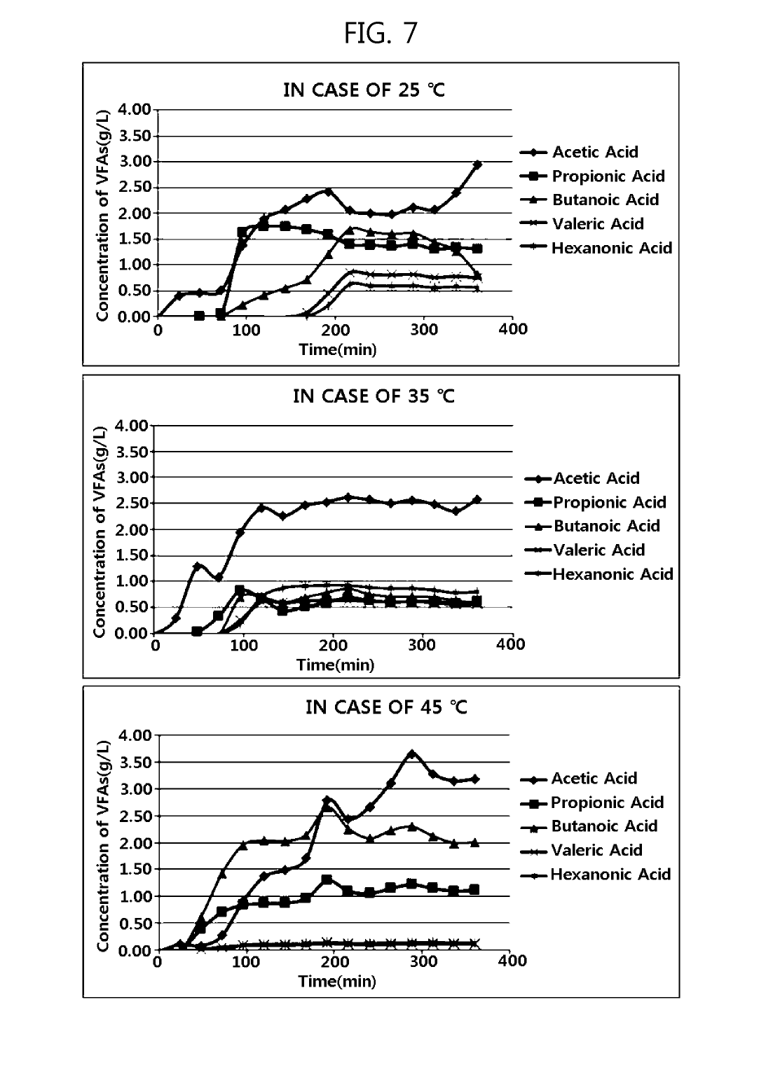

As a result, when the Laminaria japonica extract was fermented at a temperature of 35° C., the produced VFAs were produced at the highest concentration and yield, as listed in Table 11 and shown in FIG. 7. Also, for the compositions of the produced VFAs, acetic acid and propionic acid were produced at relatively high concentrations at 25° C., acetic acid was produced at a relatively high concentration at 35° C., and acetic acid and butyric acid were produced at relatively high concentrations at 45° C.

<2-7> Analysis of Concentration and Yield of VFA According to Fermentation Acidity

In order to analyze the concentration and yield of VFAs according to the fermentation acidity, the VFAs were produced while constantly maintaining all the other conditions listed in the following Table 12, except for different fermentation acidities of pH 6, pH 6.5 and pH 7.

TABLE 12

| | | | | | | | Day 5 | | Day 10 | | Day 15 | | Day 20 | |
|---|---|---|---|---|---|---|---|---|---|---|---|---|---|---|
| | | Concen- | Volume | Fermen- | | | Concen- | | Concen- | | Concen- | | Concen- | |
| | | tration | ratio | tation | | | tration | | tration | | tration | | tration | |
| Pretreat- | Kinds | (g/L) | of | temper- | | Kinds | (g/L) of | | (g/L) of | | (g/L) of | | (g/L) of | |
| ment | of | of | micro- | ature | | of | organic | Yield | organic | Yield | organic | Yield | organic | Yield |
| method | algae | extract | organism | (° C.) | pH | media | acid | (%) | acid | (%) | acid | (%) | acid | (%) |
| First acid | Laminaria | 54 | 1/15 | 35 | 6 | Medium 1 | 4.7 | 8.7 | 21.0 | 38.9 | 18.0 | 33.3 | 17.7 | 32.8 |
| catalyst | japonica | | | | 6.5 | | 3.1 | 5.7 | 13.9 | 25.7 | 16.3 | 30.2 | 19.8 | 36.7 |
| treatment | | | | | 7 | | 2.6 | 4.8 | 17.6 | 32.6 | 20.0 | 37.0 | 21.4 | 39.6 |
| method | | | | | | | | | | | | | | |

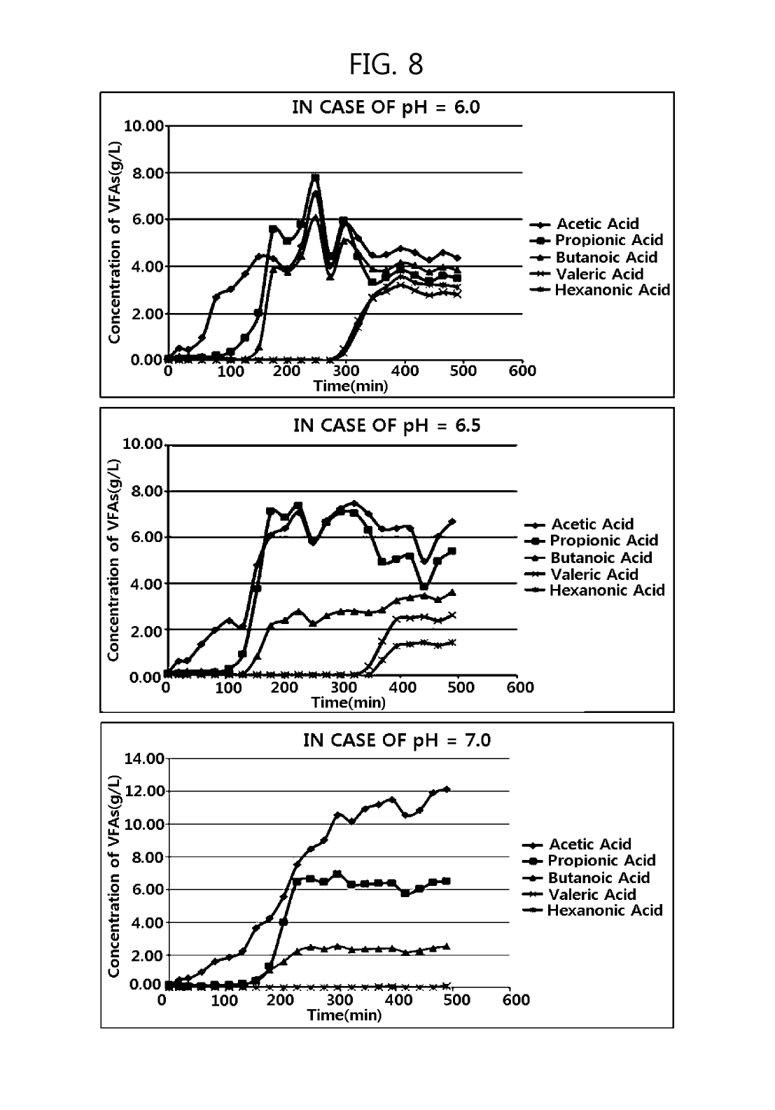

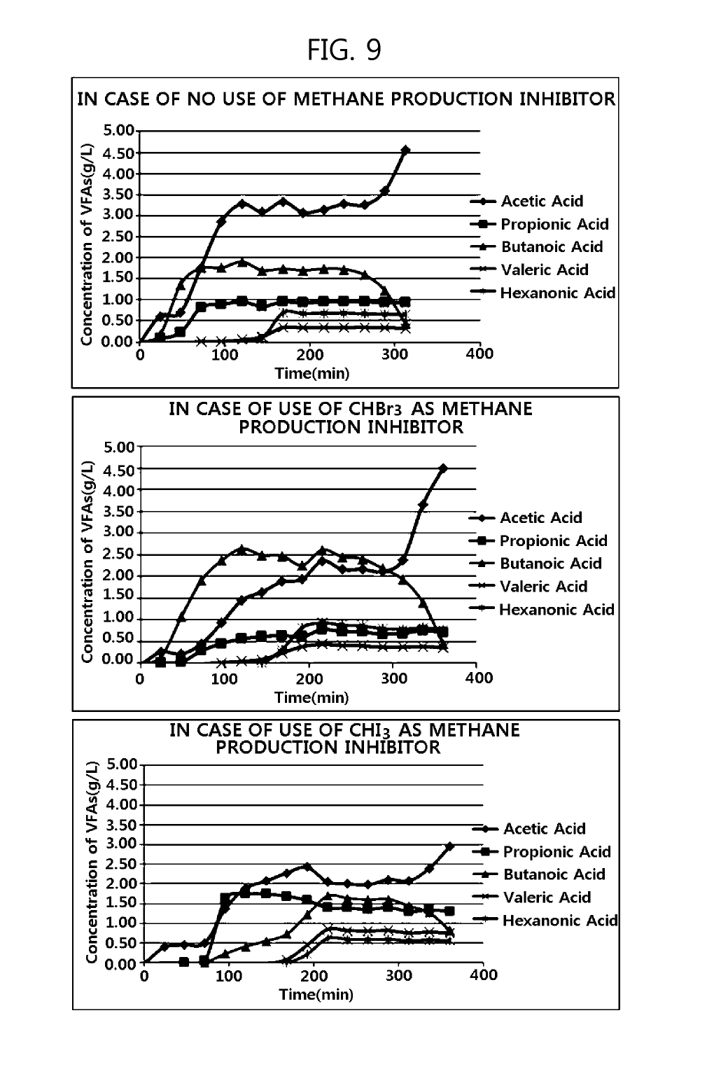

As a result, the concentration and yield of the VFAs increased according to a reaction time regardless of the fermentation acidity, and, for the compositions of the produced VFAs, acetic acid, propionic acid and butyric acid were produced at similar concentrations at pH 6, acetic acid and propionic acid were produced at relatively high concentrations at pH 6.5, and acetic acid was produced at a relatively high concentration at pH 7, as listed in Table 12 and shown in FIG. 8.

<2-8> Analysis of Concentration and Yield of VFA According to Kinds of Methane Production Inhibitor

In order to analyze the concentration and yield of VFAs according to the kinds of methane production inhibitors, the VFAs were produced while constantly maintaining all the other conditions listed in the following Table 13, except that a methane production inhibitor was not added, or different kinds of the added methane production inhibitor such as CHI₃ and CHBr₃ were used.

TABLE 13

| | | | | | | | | Day 5 | | Day 10 | | Day 15 | |
|---|---|---|---|---|---|---|---|---|---|---|---|---|---|
| | | Concen- | Volume | Fermen- | | | | Concen- | | Concen- | | Concen- | |
| | | tration | ratio | tation | | | | tration | | tration | | tration | |
| Pretreat- | Kinds | (g/L) | of | temper- | | Kinds | Methane | (g/L) of | | (g/L) of | | (g/L) of | |
| ment | of | of | micro- | ature | | of | production | organic | Yield | organic | Yield | organic | Yield |
| method | algae | extract | organism | (° C.) | pH | media | inhibitor | acid | (%) | acid | (%) | acid | (%) |
| First acid | Laminaria | 18 | 1/15 | 25 | 7 | Medium 1 | — | 6.2 | 34.4 | 7.0 | 38.9 | 6.4 | 35.6 |
| catalyst | japonica | | | | | | CHBr₃ | 4.7 | 26.1 | 6.7 | 37.2 | 6.8 | 37.8 |
| treatment | | | | | | | CHI₃ | 4.0 | 22.2 | 6.4 | 35.6 | 6.4 | 35.6 |
| method | | | | | | | | | | | | | |

As a result, the VFA was produced at relatively high speed for 5 days when there was no methane production inhibitor, and there was no significant difference in concentration of the VFAs after 10 days, as listed in Table 13. As shown in FIG. 8, acetic acid was produced at a relatively high concentration when the methane production inhibitor was not used, acetic acid and butyric acid were produced at relatively high concentrations when CHBr₃ was used, and acetic acid and propionic acid were produced at relatively high concentrations at the beginning when CHI₃ was used. The methane production inhibitor functions to prevent a reduction in concentration of the VFAs by preventing the VFAs from being converted into methane by a methane-producing microorganism during an anaerobic fermentation process.

<2-9> Analysis of Concentration and Yield of VFA According to Compositions of Fermentation Media

In order to analyze the concentration and yield of VFAs according to compositions of a fermentation medium, a microorganism was added at a volume ratio of 1/15 to 18 g/L of the extract of the pretreated algae obtained by the second acid catalyst treatment method, and anaerobically fermented at conditions of 35° C. and pH 7 in a fermentation medium including N and P at a molar ratio of 3.5:1 to produce VFAs, except that compositions of the fermentation medium varied as listed in the following Table 14.

TABLE 14

| | | | | | Day 5 | | Day 10 | | Day 15 | | Day 20 | |
|---|---|---|---|---|---|---|---|---|---|---|---|---|
| | | | | Concen- | Concen- | | Concen- | | Concen- | | Concen- | |
| | | | | tration | tration | | tration | | tration | | tration | |
| Pretreat- | Kinds | Kinds | N:P | (g/L) | (g/L) of | | (g/L) of | | (g/L) of | | (g/L) of | |
| ment | of | of | molar | of | organic | Yield | organic | Yield | organic | Yield | organic | Yield |
| method | algae | media | ratio | extract | acid | (%) | acid | (%) | acid | (%) | acid | (%) |
| Second | Laminaria | Medium 1 | 3.5:1 | 18 | 1.6 | 8.9 | 3.5 | 19.4 | 4.1 | 22.8 | 3.3 | 18.3 |
| acid | japonica | Medium 2 | | | 1.9 | 10.6 | 3.8 | 21.1 | 5.5 | 30.6 | 4.4 | 24.4 |
| catalyst | | Medium 3 | | | 1.4 | 7.8 | 3.5 | 19.4 | 4.3 | 23.9 | 5.3 | 29.4 |
| treatment | | Medium 4 | | | 1.6 | 8.9 | 3.5 | 19.4 | 4.8 | 26.7 | 5.0 | 27.8 |
| method | | Medium 5 | | | 1.0 | 5.6 | 3.1 | 17.2 | 4.7 | 26.1 | 5.2 | 28.9 |

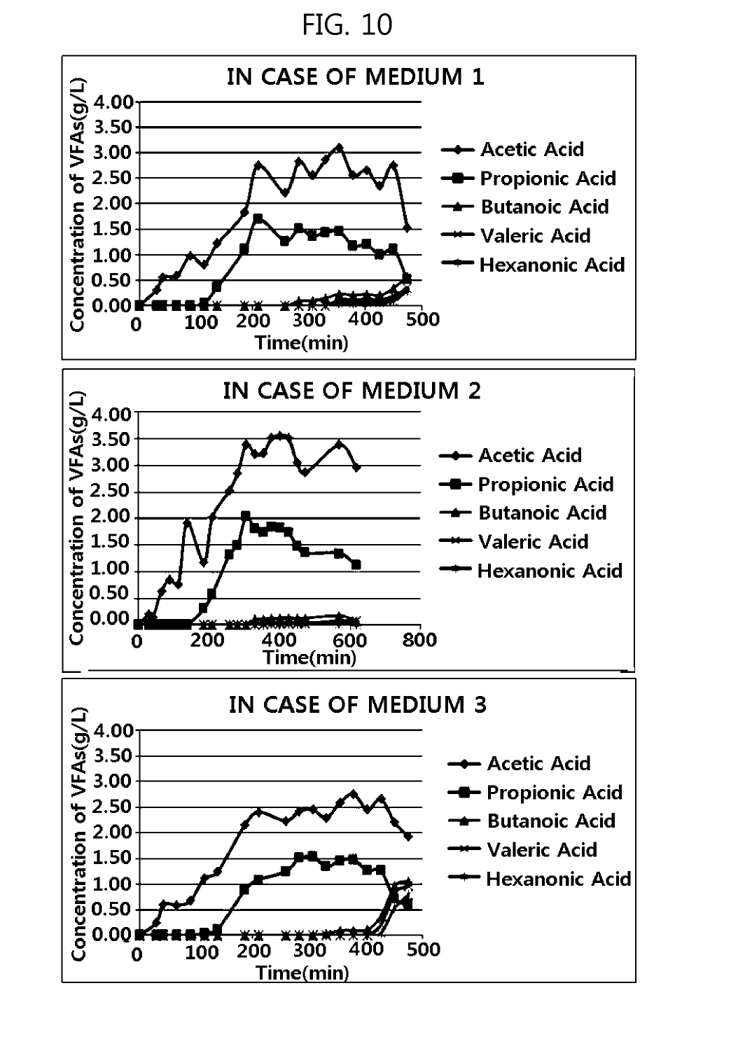

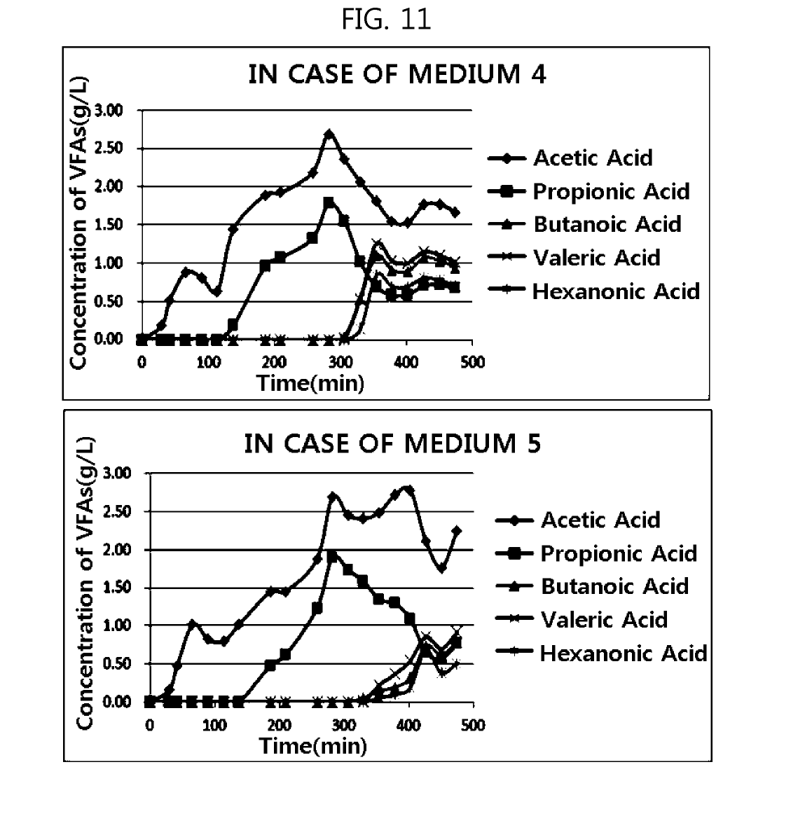

As a result, it was confirmed that a larger amount of the VFAs was produced in media 2 to 5 including two medium compositions, compared with medium 1 including various kinds of inorganic salts. As listed in Table 14 and shown in FIGS. 10 and 11, it could be seen that the VFAs were produced at relatively high concentration and yield in the medium including (NH₂)₂CO and KH₂PO₄.

<2-9> Analysis of Concentration and Yield of VFA According to N:P Ratio in Fermentation Medium

In order to analyze the concentration and yield of VFAs according to an N:P molar ratio in a fermentation medium, a microorganism and CHI₃ (a methane production inhibitor) were added at a volume ratio of 1/15 to 18 g/L of the extract of the pretreated algae obtained by the second acid catalyst treatment method, and anaerobically fermented at conditions of 35° C. and pH 7 in a medium containing (NH₂)₂CO and KH₂PO₄ to produce VFAs, except that the N:P molar ratios in the fermentation media varied as listed in the following Table 15.

TABLE 15

| | | | | | | | Day 5 | | Day 10 | | Day 15 | |
|---|---|---|---|---|---|---|---|---|---|---|---|---|
| | | | Kinds | | | Concen- | Concen- | | Concen- | | Concen- | |
| | | | of | | | tration | tration | | tration | | tration | |
| Pretreat- | Kinds | Kinds | medium | Amount | N:P | (g/L) | (g/L) of | | (g/L) of | | (g/L) of | |
| ment | of | of | compo- | added | molar | of | organic | Yield | organic | Yield | organic | Yield |
| method | algae | media | sitions | (g) | ratio | extract | acid | (%) | acid | (%) | acid | (%) |
| Second | Laminaria | 2 | (NH₂)₂CO | 0.33 | 1.5:1 | 18 | 1.2 | 6.7 | 2.7 | 15.0 | 3.5 | 19.4 |
| acid | japonica | | KH₂PO₄ | 1.00 | | | | | | | | |
| catalyst | | 2 | (NH₂)₂CO | 0.76 | 3.5:1 | 18 | 1.9 | 10.6 | 3.8 | 21.1 | 5.5 | 30.6 |
| treatment | | | KH₂PO₄ | 1.00 | | | | | | | | |
| method | | 2 | (NH₂)₂CO | 1.10 | 5.0:1 | 18 | 1.2 | 6.7 | 4.1 | 22.8 | 3.7 | 20.6 |
| | | | KH₂PO₄ | 1.00 | | | | | | | | |
| | | 2 | (NH₂)₂CO | 1.65 | 7.5:1 | 18 | 1.1 | 6.1 | 2.7 | 15.0 | 3.9 | 21.7 |
| | | | KH₂PO₄ | 1.00 | | | | | | | | |

As a result, it could be seen that the volatile organic acids were produced with high concentration and yield when the N:P molar ratio was in a range of 1.5:1 to 7.5:1, and the N:P molar ratio of 3.5:1 to 5.0:1 in the microorganism-fermenting medium was optimal, as listed in Table 15.

<2-11> Continuous Production of VFAs for Mass Production

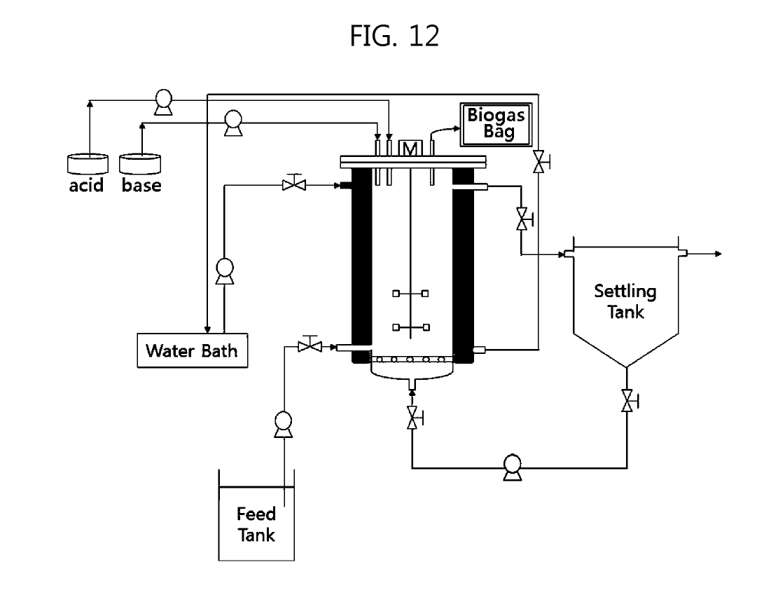

In order to continuously mass-produce the VFAs, 20 g/L of the extract of the pretreated algae obtained by the second acid catalyst treatment method, a microorganism at a volume ratio of 1/15, and medium 2 were added to a 300 L anaerobic fermentation apparatus shown in FIG. 12, and anaerobically fermented at conditions of 35° C. and pH 7 to produce VFAs, except that batch-type and continuous operating methods were used as listed in the following Table 16.

TABLE 16

| | | | | | | Day 5 | | Day 10 | | Day 15 | | Day 20 | |
|---|---|---|---|---|---|---|---|---|---|---|---|---|---|
| | | Concen- | Volume | | | Concen- | | Concen- | | Concen- | | Concen- | |
| | | tration | ratio | | Flow | tration | | tration | | tration | | tration | |
| | Pretreat- | (g/L) | of | Retention | water | (g/L) of | | (g/L) of | | (g/L) of | | (g/L) of | |
| Operating | ment | of | micro- | time | rate | organic | Yield | organic | Yield | organic | Yield | organic | Yield |
| method | method | extract | organism | (HRT) | (L/day) | acid | (%) | acid | (%) | acid | (%) | acid | (%) |
| Batch | Second | 20 | 1/30 | — | — | 6.5 | 32.5 | 6.9 | 34.5 | 6.5 | 32.5 | 5.0 | 25.0 |
| type | acid | | 1/15 | | | 4.5 | 22.5 | 6.6 | 33.0 | 4.6 | 23.0 | 4.6 | 23.0 |
| | catalyst | | 1/10 | | | 4.6 | 23.0 | 6.6 | 33.0 | 5.1 | 25.5 | 4.6 | 23.0 |
| Continuous | treatment | | 1/10 | 8 days | 12.5 | 6.7 | 33.5 | 7.2 | 36.0 | 8.5 | 42.5 | 8.6 | 43.0 |
| | method | | | | | | | | | | | | |

As a result, when the VFAs were produced on a large scale of 300 L in a batch-type manner, the VFAs were produced at a concentration similar to that of the VFAs produced on a scale of 1 L, as listed in Table 16. Thus, it was revealed that the VFAs might be produced on a pilot scale using the method according to Example embodiments of the present invention. Also, the higher productivity was realized when the VFAs were produced by means of the continuous operation, compared with when the VFAs were produced by means of the batch-type operation.

As described above, the effect of the parameters on production of the VFAs was analyzed according to the parameters according to Example embodiments of the present invention. As a result, it was confirmed that the VFAs might be produced with high efficiency according to Example embodiments of the present invention by adding a microorganism and CHI₃ (a methane production inhibitor) at a volume ratio of 1/15 to an extract filtrate having a concentration of 54 g/L, which was obtained by pretreating a dreg (residue) remaining after extracting and filtering ground brown algae, especially Laminaria japonica, using a first acid catalyst treatment method or a base catalyst treatment method, and anaerobically fermenting the extract filtrate at conditions of 35° C. and pH 7 in a medium containing (NH₂)₂CO and KH₂PO₄ (N:P molar ratio=3.5:1). The results of analysis are summarized in the following Table 17.

TABLE 17

| | | | | | | | | Day 5 | |
|---|---|---|---|---|---|---|---|---|---|
| | | | Concen- | Volume | Fermen- | | | Concen- | |
| | | | tration | ratio | tation | | | tration | |
| Pretreat- | Pretreat- | Kinds | (g/L) | of | temper- | | Kinds | (g/L) of | |
| ment | ment | of | of | micro- | ature | | of | organic | Yield |
| type | method | algae | extract | organism | (° C.) | pH | media | acid | (%) |
| Biological | Microorganism | Laminaria | 6 | 1/15 | 35 | 7 | Medium 1 | 4.4 | 73.3 |
| pretreatment | treatment | japonica | | | | | | | |
| Chemical | First acid | Laminaria | 6 | 1/15 | 35 | 7 | Medium 1 | 0.9 | 15.0 |
| pretreatment | catalyst | japonica | 18 | — | 35 | 7 | Medium 1 + CHI₃ | 3.3 | 18.3 |
| | treatment | | | 1/15 | 25 | 7 | — | 6.2 | 34.4 |
| | | | | 1/15 | 25 | 7 | Medium 1 + CHBr₃ | 4.7 | 26.1 |
| | | | | 1/15 | 25 | 7 | Medium 1 + CHI₃ | 4.0 | 22.2 |
| | | Laminaria | 18 | 1/15 | 35 | 7 | Medium 1 | 2.9 | 16.1 |
| | | japonica | | | | | | | |
| | | Pachymeniopsis | 18 | 1/15 | 35 | 7 | Medium 1 | 4.9 | 27.2 |
| | | lanceolata | | | | | | | |
| | | Enteromorpha | 18 | 1/15 | 35 | 7 | Medium 1 | 4.2 | 29.3 |
| | | crinita | | | | | | | |
| | | Laminaria | 18 | 1/30 | 45 | 7 | Medium 1 | 3.2 | 17.8 |
| | | japonica | | 1/15 | 45 | 7 | Medium 1 | 4.5 | 25.0 |
| | | | | 1/10 | 45 | 7 | Medium 1 | 4.7 | 26.1 |
| | | | 36 | 1/15 | 35 | 7 | Medium 1 | 3.8 | 10.6 |
| | | | 54 | 1/15 | 35 | 6 | Medium 1 | 4.7 | 8.7 |
| | | | | 1/15 | 35 | 6.5 | Medium 1 | 3.1 | 5.7 |
| | | | | 1/15 | 35 | 7 | Medium 1 | 2.6 | 4.8 |
| | | | 72 | 1/15 | 35 | 7 | Medium 1 | 11.6 | 16.1 |
| | Second acid | Laminaria | 18 | 1/15 | 35 | 7 | Medium 1 | 2.6 | 14.4 |
| | catalyst | japonica | | | | | | | |
| | treatment | Chlorella | 18 | 1/15 | 35 | 7 | Medium 1 | 4.2 | 29.3 |
| | Base catalyst | Laminaria | 54 | 1/15 | 35 | 7 | Medium 1 | 8.4 | 15.5 |
| | treatment | japonica | | | | | | | |
| | | Pachymeniopsis | 54 | 1/15 | 35 | 7 | Medium 1 | 7.2 | 13.3 |
| | | lanceolata | | | | | | | |
| | | Enteromorpha | 54 | 1/15 | 35 | 7 | Medium 1 | 9.6 | 17.7 |
| | | crinita | | | | | | | |
| | Supercritical | Laminaria | 18 | — | 35 | 7 | Medium 1 | 1.6 | 8.9 |
| | water | japonica | | 1/30 | 35 | 7 | Medium 1 | 3.3 | 18.3 |
| | treatment | | | 1/15 | 35 | 7 | Medium 1 | 4.9 | 27.2 |
| | | | | 1/10 | 35 | 7 | Medium 1 | 5.2 | 28.9 |
| | | | 54 | 1/15 | 35 | 7 | Medium 1 | 1.7 | 3.1 |
| | Supercritical | Laminaria | 18 | 1/30 | 35 | 7 | Medium 1 | 1.7 | 9.4 |
| | water | japonica | | 1/15 | 35 | 7 | Medium 1 | 2.1 | 11.7 |
| | treatment after | | | 1/10 | 35 | 7 | Medium 1 | 2.6 | 14.4 |
| | supercritical | | | | | | | | |
| | carbon dioxide | | | | | | | | |
| | treatment | | | | | | | | |

| | | | | | Day 10 | | Day 15 | | Day 20 | |
|---|---|---|---|---|---|---|---|---|---|---|
| | | | Concen- | Volume | Concen- | | Concen- | | Concen- | |
| | | | tration | ratio | tration | | tration | | tration | |
| Pretreat- | Pretreat- | Kinds | (g/L) | of | (g/L) of | | (g/L) of | | (g/L) of | |
| ment | ment | of | of | micro- | organic | Yield | organic | Yield | organic | Yield |
| type | method | algae | extract | organism | acid | (%) | acid | (%) | acid | (%) |
| Biological | Microorganism | Laminaria | 6 | 1/15 | 5.3 | 8.3 | 5.7 | 95.0 | 5.6 | 93.3 |
| pretreatment | treatment | japonica | | | | | | | | |
| Chemical | First acid | Laminaria | 6 | 1/15 | 2.0 | 33.3 | 2.5 | 41.7 | 2.6 | 43.3 |
| pretreatment | catalyst | japonica | 18 | — | 7.2 | 40.0 | 7.7 | 42.8 | 7.1 | 39.4 |
| | treatment | | | 1/15 | 7.0 | 38.9 | 6.4 | 35.6 | 7.3 | 40.6 |
| | | | | 1/15 | 6.7 | 37.2 | 6.8 | 37.8 | 7.1 | 39.4 |
| | | | | 1/15 | 6.4 | 35.6 | 6.4 | 35.6 | 6.6 | 36.7 |
| | | Laminaria | 18 | 1/15 | 6.8 | 37.8 | 7.6 | 42.2 | 9.0 | 50.0 |
| | | japonica | | | | | | | | |
| | | Pachymeniopsis | 18 | 1/15 | 6.6 | 36.7 | 6.0 | 33.3 | 5.6 | 31.1 |
| | | lanceolata | | | | | | | | |
| | | Enteromorpha | 18 | 1/15 | 6.5 | 36.1 | 5.3 | 29.4 | 5.0 | 27.8 |
| | | crinita | | | | | | | | |
| | | Laminaria | 18 | 1/30 | 5.2 | 28.9 | 5.6 | 31.1 | 6.0 | 33.3 |
| | | japonica | | 1/15 | 6.0 | 33.3 | 6.6 | 36.7 | 6.2 | 34.4 |
| | | | | 1/10 | 6.4 | 35.6 | 6.2 | 34.4 | 6.3 | 35.0 |
| | | | 36 | 1/15 | 12.3 | 34.2 | 14.2 | 39.4 | 15.9 | 44.2 |
| | | | 54 | 1/15 | 21.0 | 38.9 | 18.0 | 33.3 | 17.7 | 32.8 |
| | | | | 1/15 | 13.9 | 25.7 | 16.3 | 30.2 | 19.8 | 36.7 |
| | | | | 1/15 | 17.6 | 32.6 | 20.0 | 37.0 | 21.4 | 39.6 |
| | | | 72 | 1/15 | 18.1 | 25.1 | 22.3 | 31.0 | 18.8 | 26.1 |
| | Second acid | Laminaria | 18 | 1/15 | 4.5 | 25.0 | 5.1 | 28.3 | 4.3 | 23.9 |
| | catalyst | japonica | | | | | | | | |
| | treatment | Chlorella | 18 | 1/15 | 4.9 | 27.2 | 6.1 | 33.9 | 5.6 | 31.1 |
| | Base catalyst | Laminaria | 54 | 1/15 | 20.5 | 38.0 | 21.7 | 40.2 | 19.2 | 35.6 |
| | treatment | japonica | | | | | | | | |
| | | Pachymeniopsis | 54 | 1/15 | 17.2 | 31.9 | 15.2 | 28.1 | 16.0 | 29.6 |
| | | lanceolata | | | | | | | | |
| | | Enteromorpha | 54 | 1/15 | 21.9 | 40.6 | 21.0 | 38.9 | 16.9 | 31.3 |
| | | crinita | | | | | | | | |
| | Supercritical | Laminaria | 18 | — | 4.2 | 23.3 | 3.2 | 17.8 | 2.7 | 15.0 |
| | water | japonica | | 1/30 | 4.7 | 26.1 | 3.5 | 19.4 | 3.0 | 16.7 |
| | treatment | | | 1/15 | 5.2 | 28.9 | 3.5 | 19.4 | 2.8 | 15.6 |
| | | | | 1/10 | 4.7 | 26.1 | 4.0 | 22.2 | 3.5 | 19.4 |
| | | | 54 | 1/15 | 3.1 | 5.7 | 4.3 | 8.0 | 4.7 | 8.7 |
| | Supercritical | Laminaria | 18 | 1/30 | 3.1 | 17.2 | 3.1 | 17.2 | 2.0 | 11.1 |
| | water | japonica | | 1/15 | 2.8 | 15.6 | 3.6 | 20.0 | 1.5 | 8.3 |
| | treatment after | | | 1/10 | 2.8 | 15.6 | 3.3 | 18.3 | 2.6 | 14.4 |
| | supercritical | | | | | | | | | |
| | carbon dioxide | | | | | | | | | |
| | treatment | | | | | | | | | |

The method of preparing a VFA according to Example embodiments of the present invention can be useful in using a residue of an algae extract or fraction from which physiologically active materials are primarily recovered. Therefore, it is possible to enhance added value of the residue of the wasted algae, which makes it possible to secure the economical efficiency in production of biofuel or compounds. Also, the VFAs can be produced regardless of the kinds of algae, and a process of producing a VFA can be simplified since there is no need to develop an enzyme for anaerobic fermentation. In addition, since the VFAs can be produced from the other kinds of organic components such as proteins or fats, which constitute the algae, in addition to carbohydrates included in the algae, it is possible to enhance the yield of the VFAs. Furthermore, since the limited space of soils and the environmental issues can also be solved using the algae as a raw source, the method according to Example embodiments of the present invention can be useful in producing VFAs in an economical and efficient manner.

While the example embodiments of the present invention and their advantages have been described in detail, it should be understood that various changes, substitutions and alterations may be made herein without departing from the scope of the invention.

What is claimed is:

1. A method of preparing a volatile fatty acids (VFAs), comprising:
collecting a residue of algae that remains after extracting or fractionating the algae with an organic solvent;
chemically or biologically pretreating the residue of algae to obtain an extract of the algae residue;
filtering the extract of the algae residue to obtain a filtrate; and
anaerobically fermenting the filtrate.

2. The method of claim 1, wherein the algae comprise macro-algae or micro-algae.

3. The method of claim 2, wherein the macro-algae comprise at least one selected from the group consisting of brown algae, red algae and green algae.

4. The method of claim 3, wherein the brown algae comprise at least one selected from the group consisting of Laminaria japonica, Undaria pinnatifida, Hizikia fusiforme, Sargassum fulvellum, Ecklonia stolonifera and Eisenia bicyclis.

5. The method of claim 2, wherein the micro-algae comprise at least one selected from the group consisting of Chlorella, Spirulina and Dunaliella.

6. The method of claim 1, wherein the algae are ground.

7. The method of claim 1, wherein the chemical pretreatment is performed by treating the algae with at least one selected from the group consisting of an acid catalyst, an alkali catalyst, a supercritical fluid and an ionic liquid.

8. The method of claim 7, wherein the acid catalyst is at least one selected from the group consisting of sulfuric acid, nitric acid, phosphoric acid, hydrochloric acid, perchloric acid, p-toluenesulfonic acid, methanesulfonic acid, formic acid, acetic acid, hydrofluoric acid, boric acid and a commercially available solid acid.

9. The method of claim 7, wherein the alkali catalyst is at least one selected from the group consisting of potassium hydroxide, sodium hydroxide, calcium hydroxide, barium hydroxide, ammonium hydroxide and basic zeolite.

10. The method of claim 7, wherein the supercritical fluid is supercritical carbon dioxide or supercritical water.

11. The method of claim 1, wherein the biological pretreatment is performed by treating the algae with a culture broth containing abalone viscera- or foreshore-derived microorganism.

12. The method of claim 1, wherein the filtrate of the extract is obtained at a concentration of 40 g/l to 60 g/l.

13. The method of claim 1, wherein the anaerobic fermentation is performed by an anaerobic microorganism.

14. The method of claim 13, wherein the anaerobic microorganism is at least one selected from the group consisting of Clostridium sp., Acetogenium sp., Peptococcus sp., Acetobacterium sp. and Propionobacterium sp.

15. The method of claim 13, wherein the anaerobic microorganism is added at a volume ratio of 1/15 to 1/10, based on the filtrate to be fermented.

16. The method of claim 1, wherein the fermentation is performed in a medium containing at least one selected from the group consisting of inorganic salts such as ammonium salt, phosphate, calcium salt, magnesium salt and sodium salt.

17. The method of claim 1, wherein the fermentation is performed in a medium containing (NH₂)₂CO and KH₂PO₄.

18. The method of claim 16, wherein the medium contains nitrogen and phosphorus at a molar ratio of approximately 1.5:1 to 7.5:1.

19. The method of claim 1, wherein the VFA is at least one selected from the group consisting of acetic acid, propionic acid, butyric acid, valeric acid and caproic acid.

20. The method of claim 1, wherein the anaerobic fermentation is performed by adding the filtrate in a batch-type or continuous manner.
